(19) United States
(12) Patent Application Publication
(10) Pub. No.: US 2020/0377551 A1
(43) Pub. Date: Dec. 3, 2020

(54) PEPTIDE CONSTRUCTS AND METHODS FOR ENHANCEMENT OF INTERFERON PRODUCTION

(71) Applicant: Wake Forest University Health Sciences, Winston-Salem, NC (US)

(72) Inventors: Hui-Kuan Lin, Winston-Salem, NC (US); Weina Zhang, Beijing (CN); Guihua Wang, Wuhan (CN)

(73) Assignee: Wake Forest University Health Sciences, Winston-Salem, NC (US)

(21) Appl. No.: 16/882,095

(22) Filed: May 22, 2020

Related U.S. Application Data
(60) Provisional application No. 62/853,496, filed on May 28, 2019.

(51) Int. Cl. C07K 7/06; A61P 31/16

(52) U.S. Cl. CPC C07K 7/06; A61K 38/00; A61P 31/16

(57) ABSTRACT

Peptide constructs comprising a mitochondrial antiviral-signaling protein (MAVS) peptide and a cell penetration peptide are disclosed, which are useful for stimulating interferon production in vitro and in vivo. Lactate has been discovered to inhibit glycolysis-mediated retinoic acid-inducible gene I (RIG-I) like receptor signaling by directly binding to the MAVS transmembrane (TM) domain and preventing MAVS aggregation; peptide constructs according to the disclosure can prevent or reverse this inhibition to stimulate interferon production. Methods for stimulating interferon production in a cell are also described, as well as methods for the treatment of viral infections and cancer.

### CROSS-REFERENCE TO RELATED APPLICATION

The present application is a nonprovisional application of and claims the benefit of priority to U.S. Provisional Patent Application No. 62/853,496, filed May 28, 2019, the entire contents of which are incorporated by reference herein for all purposes.

### STATEMENT AS TO RIGHTS TO INVENTIONS MADE UNDER FEDERALLY SPONSORED RESEARCH AND DEVELOPMENT

This invention was made with government support under Grant Nos. R01CA182424 and R01CA193813 awarded by the National Institutes of Health. The government has certain rights in the invention.

### SEQUENCE LISTING

The official copy of the sequence listing is submitted electronically via EFS-Web as an ASCII formatted sequence listing with a file named 104134-1184874.txt, created on May 21, 2020 and having a size of 17 kilobytes, and is filed concurrently with the specification. The sequence listing contained in this ASCII formatted document is part of the specification and is herein incorporated by reference in its entirety.

### BACKGROUND

Type-I interferons (IFNs) produced by almost all type of cells play a vital role in host defense against viral infection and cancer immune-surveillance (Ivashkiv and Donlin, 2014; Zitvogel et al., 2015). Type-I IFNs composed of IFNα, IFNβ and other IFNs are induced following activation of cell-surface or intracellular pattern recognition receptors (PRRs), including retinoic acid-inducible gene I (RIG-I) like receptors (RLRs), stimulator of IFN genes protein (STING) and Toll-like receptors (TLRs) (Burdette et al., 2011; Kawai and Akira, 2010; Trinchieri, 2010). In response to microbial products (e.g., viral or bacterial components) or endogenous molecules (e.g., cytosolic and extracellular DNA and RNA), PRRs transmit distinct downstream signaling pathways to trigger type-I IFN production. Upon recognized by IFN receptors, type-I IFN signal activates the Janus kinase (JAK) signal transducer and activator of transcription (STAT) pathway, leading to the expression of IFN-stimulated genes (ISGs), which control innate and adaptive immunity and intracellular antimicrobial programs (MacMicking, 2012; Schoggins et al., 2011; Stark and Darnell, 2012).

RLRs like RIG-I and MDA5, as members of PRRs, are the main cytosolic RNA sensors to trigger innate immune response by sequentially activating downstream axis for type-I IFN production (Schlee and Hartmann, 2016; Seth et al., 2005). Upon sensing diverse cytosolic dsRNAs, RIG-I undergoes conformational changes, oligomerization and exposure of the N-terminal CARD domains to recruit a signaling adaptor called mitochondrial antiviral-signaling protein (MAVS). The transmembrane domain (TM domain) at C-terminus of MAVS is necessary for its mitochondrial outer membrane localization. Once activated, MAVS develops a functional prion-like structure at mitochondria, which serves as a platform to form a MAVS signalosome for activating TBK1 and IKKε. Phosphorylation of IRF3 and NF-κB by TBK1 and IKKε drives their nuclear translocation, leading to subsequent transcription of type-I IFN and inflammatory cytokines (Honda et al., 2006; Hou et al., 2011; Ivashkiv and Donlin, 2014; Tamura et al., 2008). Although intracellular RLR-MAVS activation and type-I IFN induction are orchestrated by numerous mechanisms (Chen et al., 2013a; Feng et al., 2017; Gack et al., 2007; Jin et al., 2017; You et al., 2013; Zhu et al., 2014), little is known about whether energy metabolism, likely through the production of a certain metabolite, is crucially involved in the regulation of this key signaling node allowing for the host defense against virus.

As a major source of cellular energy and cell mass, glucose is metabolized via glycolysis into pyruvate, which can either be imported to mitochondria for tricarboxylic acid (TCA) cycle entry to generate acetyl-CoA, through pyruvate dehydrogenase complex (PDHc) in the presence of oxygen, or be catalyzed by lactate dehydrogenase (LDH) to generate lactate when oxygen is not available. Interestingly, most cancer cells are addicted to aerobic glycolysis, known as “Warburg effect,” for their survival and proliferation even in the presence of oxygen, accompanied by high lactate generation (DeBerardinis and Thompson, 2012; Parks et al., 2013). Although lactate was previously considered to be a waste product of glucose metabolism, accumulating evidence has underscored its pivotal role in regulating diverse biological processes, such as macrophage polarization, T helper cell differentiation as well as tumor immune-surveillance (Brand et al., 2016; Colegio et al., 2014; Peng et al., 2016). Until now, no direct protein target of lactate has been identified. Whether lactate participates in other intracellular signal transduction and biological outcome through direct protein targeting remains to be determined.

### BRIEF SUMMARY

Provided herein are peptide constructs comprising a mitochondrial antiviral-signaling protein (MAVS) peptide and a cell penetration peptide. The peptide constructs are useful for preventing lactate binding to MAVS in vitro and in vivo, thereby stimulating interferon production. In some embodiments, the MAVS peptide comprises a MAVS transmembrane domain such as residues 514-535 of human MAVS protein. In some embodiments, the cell penetration peptide comprises an HIV-1 Tat sequence. Nucleic acids encoding the peptide constructs are also provided, as well as vectors and host cells containing the nucleic acids.

Also provided are methods for stimulating interferon production in a cell. The methods include contacting the cell with an effective amount of a peptide construct as described herein.

Also provided are methods for treating viral infections and cancer. The methods include administering a therapeutically effective amount of a peptide construct as described herein to a subject in need thereof. The peptide construct can be administered in conjunction with one or more antiviral agents, anti-cancer agents, hexokinase inhibitors, lactate dehydrogenase inhibitors, or a combination thereof.

### BRIEF DESCRIPTION OF THE DRAWINGS

FIG. 1A shows the quantitative analysis of some intermediates in glycolysis and TCA cycle.

FIG. 1B shows the Q-PCR analysis of IFN-β mRNA expression in HEK293 cells cultured with high glucose (25 mM) or low glucose (5 mM) and transfected with Poly(I:C)(1 μg/ml).

FIG. 1C shows Q-PCR analysis of IFN-β mRNA expression in THP1 cells transfected with HTDNA (1 μg/ml).

FIG. 1D shows Q-PCR analysis of IFN-β mRNA expression in THP1 cells stimulated with LPS (50 ng/ml).

FIG. 1E shows Q-PCR analysis of IFN-β mRNA expression from spleen tissue of mice fasted overnight and then treated with high glucose (1.5 g/kg) or low glucose (0.2 g/kg).

FIG. 1F shows Q-PCR analysis of VSV mRNA expression from spleen tissue of mice fasted overnight and then treated with high glucose (1.5 g/kg) or low glucose (0.2 g/kg).

FIG. 1G shows Q-PCR analysis of IFN-β gene expression in HEK293 cells pretreated with or without 2DG (2 mM) and then transfected with Poly(I:C) (1 μg/ml) for the indicated periods of time. Data are means±SD. *p<0.05, **p<0.01.

FIG. 1H shows Q-PCR analysis of IFN-β gene expression in HEK293 cells pretreated with or without 2DG (2 mM) and then infected with Sendai virus for the indicated periods of time. Data are means±SD. *p<0.05, **p<0.01.

FIG. 1I shows Q-PCR analysis of Sendai viral specific gene expression in HEK293 cells pretreated with or without 2DG (2 mM) and then infected with Sendai virus for the indicated periods of time. Data are means±SD. *p<0.05, **p<0.01. Collectively, FIGS. 1A-1I demonstrate that downregulation of glucose metabolism promotes RLR induced type-I IFN production.

FIG. 2A shows the analysis of hexokinase (HK) activity in purified mitochondria isolated from HEK293 cells transfected with poly(I:C).

FIG. 2B shows the Q-PCR analysis of IFN-β mRNA expression in control or HK2 knockdown Hep3B cells transfected with or without Poly(I:C).

FIG. 2C shows immunoblot analysis of Hep3B cells with control or HK2 knockdown.

FIG. 2D shows immunoblot analysis of HK2 level in the mitochondria fraction from THP1 cells transfected with Poly(I:C), HTDNA or stimulated with LPS. Tom20 was used as a mitochondria marker.

FIG. 2E shows immunoblot (IB) analysis of samples prepared by whole cell lysis of HEK293 cells infected with or without Sev followed by immunoprecipitation (IP) with IgG or MAVS antibody.

FIG. 2F shows immunoblot (IB) analysis of samples prepared by whole cell lysis of HEK293 cells infected with or without Sev followed by immunoprecipitation (IP) with IgG or MAVS antibody.

FIG. 2G shows results of experiments where HEK293 cells transfected with Flag-V or Flag-RIG-I(N) were immunoprecipitated with the indicated antibodies and IP complexes were analyzed by D3 analysis.

FIG. 2H shows results of experiments where HEK293 cells with control or RIG-I knockdown were infected with Sev for 4 hours and whole cell lysis were collected for IP with MAVS antibody, followed by IB analysis for the indicated proteins.

FIG. 2I shows the measurement of mitochondria hexokinase activity, total pyruvate level, lactate level and ECAR in Hep3B cells with control or MAVS knockdown. Data are means±SD. *p<0.05, **p<0.01.

FIG. 2J shows the measurement of mitochondria hexokinase activity, total pyruvate level, lactate level and ECAR in Hep3B cells with control or MAVS knockdown. Data are means±SD. *p<0.05, **p<0.01. Collectively, FIGS. 2A-2J show that mitochondria hexokinase activity is maintained by MAVS and inactivated during RLR activation.

FIG. 3A shows Q-PCR analysis of IFN-β mRNA expression in HEK293 cells with or without PDHA knockdown and then transfected with Poly(I:C) (left panel) or infected with Sendai virus (right panel) for the indicated times.

FIG. 3B shows Q-PCR analysis of IFN-β mRNA expression in HEK293 cells pretreated with or without DCA (10 mM) and then transfected with Poly(I:C) (left panel) or infected with Sendai virus (right panel) for the indicated times.

FIG. 3C shows Q-PCR analysis of IFN-β mRNA expression in immortalized bone marrow macrophage (iBMM) cells cultured in mediums containing glucose (25 mM) or galactose (25 mM) and then transfected with Poly(I:C) (left panel) or infected with Sendai virus (right panel) for the indicated times.

FIG. 3D shows Q-PCR analysis of IFN-β mRNA expression (left panel) and measurement of lactate secretion (right panel) in HEK293 cells exposed to normoxia (20% O₂) or hypoxia (1% O₂) and transfected with Poly(I:C).

FIG. 3E shows immunoblot analysis of HEK293 cells with control or PDHA knockdown and transfected with Poly(I:C) for the indicated times.

FIG. 3F shows immunoblot analysis of HEK293 cells with control or PDHA knockdown. Cell mitochondria were isolated for SDD-AGE (upper panel) and whole cell lysates were used for SDS-PAGE (lower panel). Data are means±SD. **p<0.01. Collectively, FIGS. 3A-3F show that anaerobic glycolysis inhibits RLR triggered MAVS-TBK1-IRF3 activation and type-I IFN production.

FIG. 4A shows the measurement of lactate secretion in Hep3B cells with control or LDHA knockdown and transfected with Poly (I:C) for 4 hours.

FIG. 4B shows the measurement of IFN-β mRNA expression in Hep3B cells with control or LDHA knockdown and transfected with Poly (I:C) for 4 hours.

FIG. 4C shows the measurement of protein levels in Hep3B cells with control or LDHA knockdown and transfected with Poly (I:C) for 4 hours.

FIG. 4D shows Q-PCR analysis of IFN-β mRNA expression in Hep3B cells infected with control or LDHA shRNA along with or without Flag-LDHA expression and then transfected with Poly (I:C).

FIG. 4E shows the measurement of lactate secretion in HEK293 cells pretreated with or without sodium oxamate (20 mM) overnight.

FIG. 4F shows Q-PCR analysis of IFN-β mRNA expression in HEK293 cells treated with or without sodium oxamate (20 mM) overnight and then transfected with Poly(I:C) for 2 hours.

FIG. 4G shows Q-PCR analysis of IFN-β mRNA expression in HEK293 cells treated with or without sodium oxamate (20 mM) overnight and infected with Sev.

FIG. 4H shows Q-PCR analysis of Sev mRNA expression in HEK293 cells treated with or without sodium oxamate (20 mM) overnight and then infected with Sev.

FIG. 4I shows Q-PCR analysis of IFN-β mRNA expression in Hep3B cells pretreated with or without sodium oxamate (20 mM) and then treated with or without Lactate (10 mM) before transfecting with Poly(I:C).

FIG. 4J shows Q-PCR analysis of IFN-β mRNA expression in Hep3B cells pretreated with or without 2-DG (2 μM) and then treated with or without Lactate (10 mM) before transfecting with Poly(I:C).

FIG. 4K shows Q-PCR analysis of IFN-β mRNA expression in Hep3B cells infected with control or HK2 shRNA and then treated with or without Lactate (10 mM) before transfecting with Poly(I:C).

FIG. 4L shows Q-PCR analysis of IFN-β mRNA expression in iBMM cells cultured in mediums containing glucose (25 mM) or galactose (25 mM) and then treated with or without Lactate before transfecting with Poly(I:C).

FIG. 4M shows Q-PCR analysis of IFN-β mRNA expression in iBMM cells cultured in mediums containing glucose (25 mM) or galactose (25 mM) and then treated with or without Lactate before transfecting with Poly(I:C).

FIG. 4N shows Q-PCR analysis of IFN-β mRNA expression in Hep3B cells with control or MCT1 knockdown and then treated with or without sodium oxamate (20 mM) overnight before lactate addition (10 mM) and poly(I:C) transfection. Data are means±SD. **p<0.01. Collectively, FIGS. 4A-4N demonstrate that LDHA-associated lactate negatively regulates RLR signaling.

FIG. 5A shows Q-PCR analysis of IFN-β and IFN-α expression in lung from mice fasted overnight and then treated with high glucose (1.5 g/kg) or low glucose (0.2 g/kg) with or without following injection of sodium lactate (1 g/kg) and infected with VSV (2×10⁷ pfu/g).

FIG. 5B shows ELISA analysis of IFN-β in sera of Ldha^{+/+} and Ldha^{-/-} mice intraperitoneal injected with VSV (2×10⁷ pfu/g).

FIG. 5C shows Q-PCR analysis of IFN-β expression in spleen from Ldha^{+/+} and Ldha^{-/-} mice infected with VSV.

FIG. 5D shows analysis of lactate secretion in supernatants of peritoneal macrophages generated from Ldha^{+/+} and Ldha^{-/-} mice and treated with Poly(I:C) transfection, Sev or VSV infection.

FIG. 5E shows ELISA analysis of IFN-β production in supernatants of peritoneal macrophages generated from Ldha^{-/-} mice and then added with or without lactate (10 mM) before VSV infection.

FIG. 5F shows Q-PCR analysis of VSV replication in different organs from Ldha^{+/+} and Ldha^{-/-} mice infected with VSV.

FIG. 5G shows analysis of IFN-β production in supernatants of peritoneal macrophages generated from Ldha^{+/+} and Ldha^{-/-} mice and treated with Poly(I:C) transfection, Sev or VSV infection.

FIG. 5H shows Q-PCR analysis of type-I IFN expression in lung from mice injected with or without sodium oxamate (750 mg/kg) and then challenged by VSV (2×10⁷ pfu/g).

FIG. 5I shows Q-PCR analysis of IFN-β mRNA expression in lung tissue from mice injected with or without sodium oxamate and infected with HSV. Data are means±SD. **p<0.01. Collectively, FIGS. 5A-5I demonstrate that LDHA-associated lactate inhibits RLR signaling in vivo.

FIG. 6A shows immunoblot analysis of binding complexes isolated from HEK293 cell extracts incubated with biotin-labeled lactate or biotin control.

FIG. 6B shows fluorescence analysis of lactate binding in complexes immunoprecipitated (IP) by IgG or anti-MAVS antibody from HEK293 cells.

FIG. 6C shows fluorescence analysis of pyruvate binding in complexes immunoprecipitated (IP) by IgG or anti-MAVS antibody from HEK293 cells.

FIG. 6D shows the immunoblot analysis of in vitro mapping assays among biotin-labeled lactate and various MAVS truncated protein translated in vitro by the TNT system.

FIG. 6E shows immunoblot analysis of in vitro pulldown assays by incubating biotin-labeled lactate or biotin control with MAVS protein translated in vitro by the TNT system along with control or TM peptide.

FIG. 6F shows a schematic of the immunoblot analysis of in vitro mapping assays among biotin-labeled lactate and various MAVS truncated protein translated in vitro by the TNT system.

FIG. 6G shows the analysis of lactate binding with different doses of Tat-control or Tat-TM peptide.

FIG. 6H shows Q-PCR analysis of IFN-β expression in Hep3B cells treated by control or TM peptide of MAVS and transfected with Poly(I:C).

FIG. 6I shows Q-PCR analysis of IFN-β expression in Hep3B cells pretreated with or without sodium oxamate overnight, then incubated with control or TM peptide of MAVS for 2 hours and addition of lactate, followed by transfection of Poly(I:C). Data are means±SD. *p<0.05, **p<0.01. Collectively, FIGS. 6A-6I demonstrate that LDHA-associated lactate negatively regulates RLR activation by targeting MAVS.

FIG. 7A shows immunoblot analysis of the mitochondria fraction isolated from HEK293 cells treated as indicated.

FIG. 7B shows the results obtained when cell lysates from HEK293 cells transfected or treated as indicated were immunoprecipitated with antibodies indicated, and IP complexes were analyzed by immunoblot analysis.

FIG. 7C shows the results obtained when cell lysates from HEK293 cells transfected or treated as indicated were immunoprecipitated with the indicated antibodies, and IP complexes were analyzed by immunoblot analysis.

FIG. 7D shows the results obtained when cell lysates from HEK293 cells transfected or treated as indicated were immunoprecipitated with the indicated antibodies, and IP complexes were analyzed by immunoblot analysis.

FIG. 7E shows immunoblot analysis of in vitro MAVS aggregation. GST-RIG-I(N) was incubated with K63-Ub4 and then with mitochondria isolated from HEK293 cells preincubated with or without lactate (5 mM, 10 mM) or pyruvate, followed by analysis of mitochondria extracts using SDD-AGE (left panel) and SDS-PAGE (right panel). GST-RIG-(N) was shown by Coomassie blue staining (CBB).

FIG. 7F shows an illustration of how glycolysis-derived lactate inhibits RLR signaling by targeting MAVS. Collectively, FIGS. 7A-7G demonstrate that lactate inhibits RIG-I/MAVS association and MAVS aggregation.

### DETAILED DESCRIPTION

RLR-mediated Type-I IFN production plays a pivotal role in elevating host immunity for viral clearance and cancer immune-surveillance. The invention described in this disclosure is based in part on the discovery that glycolysis, which is inactivated during RLR activation, serves as a barrier to impede type-I IFN production upon RLR activation. RLR-triggered MAVS-RIG-I recognition hijacks hexokinase binding to MAVS, leading to the impairment of hexokinase localization to mitochondria and activation. Lactate serves as a key metabolite responsible for glycolysis-mediated RLR signaling inhibition by directly binding to the MAVS transmembrane (TM) domain and preventing MAVS aggregation. Notably, lactate restoration reverses increased IFN production caused by lactate deficiency. The pharmacological and genetic approaches described herein show that lactate reduction by LDHA inactivation heightens type-I IFN production to protect mice from viral infection. This study establishes a critical role of glycolysis-derived lactate in limiting RLR signaling and identifies MAVS as a direct sensor of lactate, which functions to connect energy metabolism and innate immunity.

### I. Definitions

The terms “peptide,” “polypeptide,” and “protein” are used interchangeably herein to refer to a polymer of amino acid residues linked by covalent peptide bonds. All three terms apply to naturally occurring amino acid polymers and non-natural amino acid polymers, as well as to amino acid polymers in which one (or more) amino acid residue is an artificial chemical mimetic of a corresponding naturally occurring amino acid. Unless otherwise specified, the terms encompass amino acid chains of any length, including full-length proteins.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

The terms “nucleic acid,” “nucleotide,” and “polynucleotide” refer to deoxyribonucleic acids (DNA) or ribonucleic acids (RNA) and polymers. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), orthologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“Percentage of sequence identity” is determined by comparing two optimally aligned sequences over a comparison window, wherein the portion of the sequence (e.g., a peptide of the invention) in the comparison window may comprise additions or deletions (i.e., gaps) as compared to the reference sequence which does not comprise additions or deletions, for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

“Identical” and “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection. These definitions also refer to the complement of a nucleic acid test sequence.

“Similarity” and “percent similarity,” in the context of two or more polypeptide sequences, refer to two or more sequences or subsequences that have a specified percentage of amino acid residues that are either the same or similar as defined by a conservative amino acid substitutions (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% similar over a specified region), when compared and aligned for maximum correspondence over a comparison window, or designated region as measured using one of the following sequence comparison algorithms or by manual alignment and visual inspection. Sequences are “substantially similar” to each other if, for example, they are at least 20%, at least 25%, at least 30%, at least 35%, at least 40%, at least 45%, at least 50%, or at least 55% similar to each other.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA, 90: 5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

As used herein, the terms “mitochondrial antiviral-signaling protein” and “MAVS protein” refer to the protein product of the MAVS gene in humans (GenBank Accession No. DQ174270.1) and homologous genes in other species. The term “MAVS peptide” refers to any portion of the MAVS amino acid sequence other than the full-length sequence. The term “transmembrane domain” references to a hydrophobic amino acid sequence that targets MAVS to the mitochondrial membrane (e.g., residues of 514-535 of human MAVS).

As used herein, the term “cell penetration peptide” refers to an amino acid sequence that, when linked to a second peptide (e.g., a MAVS peptide), causes or enhances the ability of the second peptide to cross the cell membrane of a cell when the cell is contacted by the cell penetration peptide linked to the second peptide.

As used here, the terms “interferon” and “IFN” refer to a family of secreted proteins produced by a variety of eukaryotic cells upon exposure to various environmental stimuli, including virus infection or exposure to a mitogen. IFNs can elicit many changes in cellular behavior, including effects on cellular growth and differentiation and modulation of the immune system. Human IFNs have been classified into subgroups including IFN-α, IFN-β, IFN-γ, IFN-ω, IFN-ε and IFN-κ. IFN-α (leukocyte-derived interferon) is produced in human leukocyte cells and, together with minor amounts of HuIFN-β (fibroblast-derived interferon), in lymphoblastoid cells. Interferons have been further classified by their chemical and biological characteristics into two general categories: Type I and Type II. Type-I interferons include IFN-α and INF-β subgroups as well as IFN-ω, IFN-ε and IFN-κ subgroups, while the only type-II interferon is IFN-γ.

As used herein, the term “hexokinase” refers to a family of enzymes that catalyze the phosphorylation of a six-carbon sugar, a hexose, to yield a hexose phosphate as the product, in the presence of ATP. There are four mammalian hexokinase isoforms, numbered I-IV, as classified as EC 2.7.1.1. Hexokinase II is the predominant form found in skeletal muscle and is located in the mitochondrial outer membrane. Hexokinase II catalyzes the formation of glucose 6-phosphate first step of glucose metabolism.

As used herein, the terms “lactate dehydrogenase” and “LDH” refer to an enzyme that catalyzes the conversion of pyruvate to lactate. Lactate dehydrogenases are classified as EC 1.1.1.27 (L-lactate dehydrogenase) or EC 1.1.1.28 (D-lactate dehydrogenase). Lactose dehydrogenase is a tetramer, most often containing 1-4 LDH-M monomers (encoded by the LDHA gene) and 1-4 LDH-H monomers (encoded by the LDHB gene).

As used herein, the term “viral infection” refers to the introduction of a virus, e.g., a parainfluenza virus, into cells or tissues. In general, the introduction of a virus is also associated with replication. Viral infection may be determined by measuring virus antibody titer in samples of a biological fluid, such as blood, using, e.g., enzyme immunoassay. Other suitable diagnostic methods include molecular based techniques, such as RT-PCR, direct hybrid capture assay, nucleic acid sequence based amplification, and the like. A virus may infect an organ, e.g., the lungs or the liver, and cause disease, e.g., pneumonia, hepatitis, or chronic liver disease.

As used herein, the term “cancer” refers to a neoplasm or tumor resulting from abnormal uncontrolled growth of cells. The term “cancer” encompasses disease states involving pre-malignant and/or malignant cancer cells. The cancer may be a localized overgrowth of cells that has not spread to other parts of a subject (e.g., a benign tumor), or the cancer may be have reached varying stages of invasion/destruction of neighboring body structures and spreading to distant sites (e.g., a malignant tumor).

As used herein, the terms “treat,” “treatment,” and “treating” refer to any indicia of success in the treatment or amelioration of an injury, pathology, condition, or symptom (e.g., lung cancer), including any objective or subjective parameter such as abatement; remission; diminishing of symptoms or making the symptom, injury, pathology or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, e.g., the result of a physical examination.

As used herein, the terms “effective amount” and “therapeutically effective amount” refer to a dose of a compound such as a cyclic dinucleotide that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, Ed., McGraw-Hill; and Remington: The Science and Practice of Pharmacy, 21^{st} Edition, 2005, Hendrickson, Ed., Lippincott, Williams & Wilkins).

As used herein, the term “subject” refers to animals such as mammals, including, but not limited to, primates (e.g., humans), cows, sheep, goats, horses, dogs, cats, rabbits, rats, mice and the like.

As used herein, the term “pharmaceutically acceptable excipient” refers to a substance that aids the administration of an active agent to a subject. By “pharmaceutically acceptable,” it is meant that the excipient is compatible with the other ingredients of the formulation and is not deleterious to the recipient thereof. Pharmaceutical excipients useful in the compositions include, but are not limited to, binders, fillers, disintegrants, lubricants, glidants, coatings, sweeteners, flavors and colors.

As used herein, the term “salt” refers to acid or base salts of active ingredients or other components. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Pharmaceutically acceptable salts of acidic compounds are salts formed with bases, namely cationic salts such as alkali and alkaline earth metal salts (such as sodium, lithium, potassium, calcium, and magnesium salts), as well as ammonium salts (such as ammonium, trimethyl-ammonium, diethylammonium, and tris-(hydroxymethyl)-methyl-ammonium salts). The neutral forms of the compounds can be regenerated by contacting the salt with a base or acid.

As used herein, the terms “about” and “around” indicate a close range around a numerical value when used to modify that specific value. If “X” were the value, for example, “about X” or “around X” would indicate a value from 0.9X to 1.1X, e.g., a value from 0.95X to 1.05X, or a value from 0.98X to 1.02X, or a value from 0.99X to 1.01X. Any reference to “about X” or “around X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

### II. Peptide Constructs

Provided herein are peptide constructs comprising a mitochondrial antiviral-signaling protein (MAVS) peptide and a cell penetration peptide. The human MAVS gene (GenBank Accession No. DQ174270.1) is conserved in chimpanzees, Rhesus monkeys, dogs, cows, mice, and rats, and ortholog genes have been identified in over 200 other organisms. The gene product (GenBank Accession No. AAZ80417.1) is a 540-residue protein containing an N-terminal caspase recruitment domain (CARD), an unstructured loop with proline-rich region, and a C-terminal transmembrane (TM) domain. The TM domain is understood to be necessary localization of MAVS in the mitochondrial outer membrane. Activation of MAVS by RIG-I leads to transcription of type-I IFN, and lactate binding to the MAVS TM domain has now been discovered to prevent RIG-I binding to MAVS as described in more detail below. In some embodiments, the MAVS peptide comprises a MAVS transmembrane domain corresponding to residues 514-535 of SEQ ID NO:1, or a sequence having at least 70% identity (e.g., at least 70%, 75%, 80%, 85%, 90%, 95%, or 99% identity) thereto. In some embodiments, the MAVS transmembrane domain comprises an amino acid sequence having at least 70% identity to SEQ ID NO:2, from MAVS in humans. In some embodiments, the MAVS transmembrane domain comprises an amino acid sequence having at least 70% identity to SEQ SEQ ID NO:3, MAVS in N. leucogenys, H. albibarbis, and S. syndactylus. In some embodiments, the MAVS transmembrane domain comprises an amino acid sequence having at least 70% identity to SEQ ID NO:4, from MAVS in M. mulatta, P. anubis, and T. gelada. In some embodiments, the MAVS transmembrane domain comprises an amino acid sequence having at least 70% identity to SEQ ID NO:5, from MAVS in C. capucinus imitator and C. jacchus. In some embodiments, the MAVS transmembrane domain comprises an amino acid sequence having at least 80% identity to SEQ ID NO:2, SEQ ID NO:3, SEQ ID NO:4, or SEQ ID NO:5. In some embodiments, the MAVS transmembrane domain comprises an amino acid sequence having at least 90% identity to SEQ ID NO:2, SEQ ID NO:3, SEQ ID NO:4, or SEQ ID NO:5.

In some embodiments, the MAVS peptide may contain one or more amino acid substitutions, deletions, or additions with respect to a wild-type MAVS sequence. Certain substitutions will be recognized as “conservative” modification where the alteration results in the substitution of an amino acid with a chemically similar amino acid. Chemically similar amino acids may include, without limitation, genetically-encoded amino acids such as an L-amino acids, stereoisomers of genetically-encoded amino acids such as a D-amino acids, N-substituted amino acids (e.g., N-methylglycine), amino acid analogs, amino acid mimetics, and synthetic amino acids. Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another (see, e.g., Creighton, Proteins, 1993):

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

In some embodiments, the MAVS peptide comprises the sequence set forth in SEQ ID NO:2, SEQ ID NO:3, SEQ ID NO:4, or SEQ ID NO:5 and one or more amino acid substitutions (e.g., conservative substitutions). For example, one or more conservative substitution or non-conservative substitutions may be made at positions 2, 3, 14, 15, 17, 29, 20, or 21 of SEQ ID NO:2, SEQ ID NO:3, SEQ ID NO:4, or SEQ ID NO:5.

The peptide constructs provided herein also contain one or more cell penetration peptides linked to the MAVS peptide (e.g., to the N-terminus of the MAVS peptide or the C-terminus of the MAVS peptide). In some embodiments, the C-terminus of the cell penetration peptide is linked to the N-terminus of the MAVS peptide. A number of cell penetration peptides can be linked to the MAVS peptide so as to enhance delivery of the MAVS peptide to target cells in vitro and/or in vivo (see, e.g., Guidotti 2017). In some embodiments, the cell penetration peptide is a cationic peptide having 5-25 total amino acid residues and at least 5 arginine residues, lysine residues, or a combination thereof. In some embodiments, the cell penetration peptide is a polyarginine ranging in length from 5 residues to 25 residues. In some embodiments, the cell penetration peptide comprises an amino acid sequence set forth in Table 1 or a sequence having at least 70% identity (e.g., at least 70%, 75%, 80%, 85%, 90%, 95%, or 99% identity) thereto.

TABLE 1
Cell Penetration Peptides

| SEQ | | |
|---|---|---|
| ID NO: | Sequence | Origin |
| 6 | YGRKKRRQRRRA | HIV-1 Tat protein |
| 7 | GRKKRRQRRRPPQ | HIV-1 Tat protein |
| 8 | RKKRRQRRR | HIV-1 Tat protein |
| 9 | RQIKIWFQNRRMKWKK | Antennapedia |
| | | Drosophila |
| | | melanogaster |
| 10 | VKRGLKLRHVRPRVTRMDV | Chemically |
| | | synthesized |
| 11 | GALFLGFLGAAGSTMGAWS | HIV glycoprotein |
| | QPKKKRKV | 41/SV40 T antigen |
| | | NLS |
| 12 | KETWWETWWTEWSQPKK | Tryptophan-rich |
| | KRKV | cluster/SV40 T |
| | | antigen NLS |
| 13 | LLIILRRRIRKQAHAHSK | Vascular |
| | | endothelial |
| | | cadherin |
| 14 | MVRRFLVTLRIRRACGPPR | p14ARF protein |
| | VRV | |
| 15 | MVKSKIGSWILVLFVA | N terminus of |
| | MWSDVGLCKKRP | unprocessed bovine |
| | | prion protein |
| 16 | KLALKLALKALKAALKLA | Chemically |
| | | synthesized |
| 17 | GWTLNSAGYLLGKINLKALA | Chimeric galanin- |
| | ALAKKIL | mastoparan |
| 18 | LSTAADMQGVVTDGMASGL | Azurin |
| | DKDYLKPDD | |
| 19 | DPKGDPKGVTVTVTVTVT | Synthetic |
| | GKGDPKPD | |
| 20 | RRIRPRPPRLPRPRPRPLPF | Bactenecin family |
| | PRPG | of antimicrobial |
| | | peptides |
| 21 | CSIPPEVKFNKPFVYLI | α1-Antitrypsin |
| 22 | PFVYLI | Derived from |
| | | synthetic C105Y |
| 23 | SDLWEMMMVSLACQY | CHL8 peptide |
| | | phage clone |

In some embodiments, the cell penetration peptide is an HIV-1 Tat peptide (e.g., a peptide according to SEQ ID NO: 6, 7, or 8). In some embodiments, the HIV-1 Tat peptide comprises an amino acid sequence having at least 70% identity to SEQ ID NO:6. In some embodiments, the HIV-1 Tat peptide comprises an amino acid sequence having at least 80% identity to SEQ ID NO:6. In some embodiments, the HIV-1 Tat peptide comprises an amino acid sequence having at least 90% identity to SEQ ID NO:6. In some embodiments, the peptide construct comprises a cell penetration peptide having the amino acid sequence according to SEQ ID NO:6 and a MAVS peptide comprising an amino acid sequence having at least 70% identity (e.g., at least 80% identity, or at least 90% identity) to SEQ ID NO:2. In some embodiments, the peptide construct comprises an amino acid sequence according to SEQ ID NO:24, or a sequence having at least 70% identity (e.g., at least 80% identity, or at least 90% identity) thereto.

In a related aspect, the present disclosure provides nucleic acids encoding peptide constructs as described herein. The nucleic acids can be generated from a nucleic acid template encoding a MAVS protein, using any of a number of known recombinant DNA techniques. Accordingly, certain embodiments of the present disclosure provide an isolated nucleic acid comprising a polynucleotide sequence encoding a peptide construct comprising a MAVS peptide and a cell penetration peptide (including, but not limited to, peptide constructs comprising amino acid sequences having at least 70%, 80%, or 90% identity to SEQ ID NO:24). Using these nucleic acids, a variety of expression constructs and vectors can be made. Generally, expression vectors include transcriptional and translational regulatory nucleic acid regions operably linked to the nucleic acid encoding the MAVS peptide construct. Control sequences that are suitable for prokaryotes, for example, include a promoter, optionally an operator sequence, and a ribosome binding site. In addition, the vector may contain a Positive Retroregulatory Element (PRE) to enhance the half-life of the transcribed mRNA (see, Gelfand et al. U.S. Pat. No. 4,666,848). The transcriptional and translational regulatory nucleic acid regions will generally be appropriate to the host cells used to express the peptide constructs. Numerous types of appropriate expression vectors and suitable regulatory sequences are known in the art for a variety of host cells. In general, the transcriptional and translational regulatory sequences may include, e.g., promoter sequences, ribosomal binding sites, transcriptional start and stop sequences, translational start and stop sequences, and enhancer or activator sequences. Typically, the regulatory sequences will include a promoter and/or transcriptional start and stop sequences. Vectors also typically include a polylinker region containing several restriction sites for insertion of foreign DNA. Heterologous sequences (e.g., a fusion tag such as a His tag) can be used to facilitate purification and, if desired, removed after purification. The construction of suitable vectors containing DNA encoding replication sequences, regulatory sequences, phenotypic selection genes, and MAVS peptide constructs can be prepared using standard recombinant DNA procedures. Isolated plasmids, viral vectors, and DNA fragments can be cleaved, tailored, and ligated together in a specific order to generate the desired vectors, as is well-known in the art (see, e.g., Green and Sambrook, Molecular Cloning: A Laboratory Manual (Cold Spring Harbor Laboratory Press, New York, 4^{th} ed. 2012)).

Provided in some embodiments is an expression cassette comprising a nucleic acid encoding a MAVS peptide construct as described herein operably linked to a promoter. In some embodiments, a vector comprising a nucleic acid encoding the MAVS peptide construct is provided. In certain embodiments, the expression vector contains a selectable marker gene to allow the selection of transformed host cells. Selection genes are well known in the art and will vary with the host cell used. Suitable selection genes can include, for example, genes coding for ampicillin and/or tetracycline resistance, which enables cells transformed with these vectors to grow in the presence of these antibiotics.

In some embodiments, a nucleic acid encoding a MAVS peptide construct is introduced into a cell, either alone or in combination with a vector. By “introduced into,” it is meant that the nucleic acids enter the cells in a manner suitable for subsequent integration, amplification, and/or expression of the nucleic acid. The method of introduction is largely dictated by the targeted cell type. Exemplary methods include CaPO₄ precipitation, liposome fusion, LIPOFECTIN®, electroporation, heat shock, viral infection, and the like.

In some embodiments, prokaryotes are used as host cells for initial cloning steps. Other host cells include, but are not limited to, eukaryotic (e.g., mammalian, plant and insect cells), or prokaryotic (bacterial) cells. Exemplary host cells include, but are not limited to, Escherichia coli, Saccharomyces cerevisiae, Pichia pastoris, Sf9 insect cells, and CHO cells. They are particularly useful for rapid production of large amounts of DNA, for production of single-stranded DNA templates used for site-directed mutagenesis, for screening many constructs simultaneously, and for DNA sequencing of the constructs generated. Suitable prokaryotic host cells include E. coli K12 strain 94 (ATCC No. 31,446), E. coli strain W3110 (ATCC No. 27,325), E. coli K12 strain DG116 (ATCC No. 53,606), E. coli X1776 (ATCC No. 31,537), and E. coli B; and other strains of E. coli, such as HB101, JM101, NM522, NM538, and NM539. Many other species and genera of prokaryotes including bacilli such as Bacillus subtilis, other enterobacteriaceae such as Salmonella typhimurium or Serratia marcesans, and various Pseudomonas species can all be used as hosts. Prokaryotic host cells or other host cells with rigid cell walls are typically transformed using the calcium chloride method as described in Green and Sambrook et al., supra. Alternatively, electroporation can be used for transformation of these cells. Prokaryote transformation techniques are set forth in, for example Dower, in Genetic Engineering, Principles and Methods 12:275-296 (Plenum Publishing Corp., 1990); Hanahan et al., Meth. Enzymol., 204:63, 1991. Plasmids typically used for transformation of E. coli include pBR322, pUCI8, pUCI9, pUCI18, pUC119, and Bluescript M13, all of which are described by Green and Sambrook et al., supra. However, many other suitable vectors are available as well.

Accordingly, some embodiments of the present disclosure provide a host cell comprising a nucleic acid encoding a MAVS peptide construct, an expression cassette, or a vector as described herein. The host cells can be prokaryotic or eukaryotic. The host cells can be mammalian, plant, bacteria, or insect cells. In some embodiments, a MAVS peptide construct is produced by culturing a host cell transformed with an expression vector containing a nucleic acid encoding MAVS peptide construct, under the appropriate conditions to induce or cause expression of the MAVS peptide construct. Methods of culturing transformed host cells under conditions suitable for protein expression are well-known in the art (see, e.g., Green and Sambrook et al., supra). Suitable host cells for production of the peptide constructs from lambda pL promoter-containing plasmid vectors include E. coli strain DG116 (ATCC No. 53606) (see U.S. Pat. No. 5,079,352 and Lawyer, F. C. et al., PCR Methods and Applications 2:275-87, 1993, which are both incorporated herein by reference). Suitable host cells for production of the MAVS peptide constructs from T7 promoter-containing plasmid vectors include E. coli strain BL21 (DE3) and related lysogens (see, e.g., U.S. Pat. No. 5,693,489). Following expression, a MAVS peptide construct can be harvested and isolated.

Alternatively, MAVS peptide constructs as described herein may be synthesized by solid-phase peptide synthesis methods, during which N-α-protected amino acids having protected side chains are added stepwise to a growing polypeptide chain linked by its C-terminus to a solid support, e.g., polystyrene beads. Various chemistries, resins, protecting groups, protected amino acids and reagents can be employed as described, for example, by Barany and Merrifield, “Solid-Phase Peptide Synthesis,” in The Peptides: Analysis, Synthesis, Biology Gross and Meienhofer (eds.), Academic Press, N.Y., vol. 2, pp. 3-284 (1980); Atherton et al., Solid Phase Peptide Synthesis: A Practical Approach, IRL Press (1989); Bodanszky, Peptide Chemistry, A Practical Textbook, 2nd Ed., Springer-Verlag (1993)); and Chan et al. Fmoc Solid Phase Peptide Synthesis: A Practical Approach, Oxford University Press (2000).

Non-limiting examples of support materials for solid-phase peptide synthesis include polystyrene (e.g., microporous polystyrene resin, mesoporous polystyrene resin, macroporous polystyrene resin; including commercially-available Wang resins, Rink amide resins, and trityl resins), glass, polysaccharides (e.g., cellulose, agarose), polyacrylamide resins, polyethylene glycol, or copolymer resins (e.g., comprising polyethylene glycol, polystyrene, etc.). The solid support may have any suitable form factor. For example, the solid support can be in the form of beads, particles, fibers, or in any other suitable form factor. Non-limiting examples of protecting groups (e.g., N-terminal protecting groups) include Fmoc, Boc, allyloxycarbonyl (Alloc), benzyloxycarbonyl (Z), and photolabile protecting groups. Sidechain protecting groups include, but are not limited to, Fmoc; Boc; cyclohexyloxycarbonyl (Hoc); allyloxycarbonyl (Alloc); mesityl-2-sulfonyl (Mts); 4-(N-methylamino)butanoyl (Nmbu); 2,4-dimethylpent-3-yloxycarbonyl (Doc); 1-(4,4-dimethyl-2,6-dioxocyclohex-1-ylidene)-3-ethyl (Dde); 1-(4,4-dimethyl-2,6-dioxocyclohex-1-ylidene)-3-methylbutyl (ivDde); 4-methyltrityl (Mtt). Additional protecting groups and methods for their addition and removal from supported peptides are described, for example, by Isidro-Llobet et al. Chem. Rev. 2009, 19: 2455-2504.

A base may be used to activate or complete the activation of amino acids prior to exposing the amino acids to immobilized peptides. In some embodiments, the base is a non-nucleophilic bases, such as triisopropylethylamine, N,N-diisopropylethylamine, certain tertiary amines, or collidine, that are non-reactive to or react slowly with protected peptides to remove protecting groups. A coupling agent may be used to form a bond with the C-terminus of an amino acid to facilitate the coupling reaction and the formation of an amide bond. The coupling agent may be used to form activated amino acids prior to exposing the amino acids to immobilized peptides. Any suitable coupling agent may be used. In some embodiments, the coupling agent is a carbodiimide, a guanidinium salt, a phosphonium salt, or a uronium salt. Examples of carbodiimides include, but are not limited to, N,N′-dicyclohexylcarbodiimide (DCC), 1-ethyl-3-(3-dimethylaminopropyl)carbodiimide (EDC), and the like. Examples of phosphonium salts include, but are not limited to, such as (benzotriazol-1-yloxy)tripyrrolidinophosphonium hexafluorophosphate (PyBOP); bromotris(dimethylamino)phosphonium hexafluorophosphate (BroP); and the like. Examples of guanidinium/uronium salts include, but are not limited to, O-(benzotriazol-1-yl)-N,N,N′,N′-tetramethyluronium hexafluorophosphate (HBTU); 2-(7-aza-1H-benzotriazole-1-yl)-1,1,3,3-tetramethyluronium hexafluorophosphate (HATU); 1-[(1-(cyano-2-ethoxy-2-oxoethylideneaminooxy) dimethylaminomorpholino)] uronium hexafluorophosphate (COMU); and the like.

### III. Pharmaceutical Compositions

Some embodiments of the present disclosure provide pharmaceutical compositions containing one or more MAVS peptide constructs as described herein and one or more pharmaceutically acceptable excipients. The pharmaceutical compositions can be prepared by any of the methods well known in the art of pharmacy and drug delivery. In general, methods of preparing the compositions include the step of bringing a MAVS peptide construct and any other active ingredients into association with a carrier containing one or more accessory ingredients. The pharmaceutical compositions are typically prepared by uniformly and intimately bringing the active ingredient(s) into association with a liquid carrier or a finely divided solid carrier or both, and then, if necessary, shaping the product into the desired formulation. The compositions can be conveniently prepared and/or packaged in unit dosage form. In addition to the MAVS peptide constructs, pharmaceutical compositions provided herein may also contain additional active ingredients such as anti-viral agents, anti-cancer agents, hexokinase inhibitors, and lactate hydrogenase inhibitors as described below.

Pharmaceutical compositions containing MAVS peptide constructs can be in the form of aqueous or oleaginous solutions and suspensions (e.g., sterile injectable solutions for intravenous, intraperitoneal, intramuscular, intralesional, subcutaneous, or intrathecal injection; or sterile solutions or suspensions for administration as a nasal spray or nasal drops). Such preparations can be formulated using non-toxic parenterally-acceptable vehicles including water, Ringer's solution, and isotonic sodium chloride solution, and acceptable solvents such as 1,3-butane diol. In addition, sterile, fixed oils can be used as a solvent or suspending medium. For this purpose any bland fixed oil can be employed including synthetic monoglycerides, diglycerides, or triglycerides.

Aqueous suspensions can contain one or more MAVS peptides in admixture with excipients including, but not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate. Dispersible powders and granules (suitable for preparation of an aqueous suspension by the addition of water) can contain one or more MAVS peptide constructs in admixture with a dispersing agent, wetting agent, suspending agent, or combinations thereof. Oily suspensions can be formulated by suspending a MAVS peptide construct in a vegetable oil (e.g., arachis oil, olive oil, sesame oil or coconut oil), or in a mineral oil (e.g., liquid paraffin). Oily suspensions can contain one or more thickening agents, for example beeswax, hard paraffin, or cetyl alcohol. These compositions can be preserved by the addition of an anti-oxidant such as ascorbic acid.

Pharmaceutical compositions containing MAVS peptide constructs can also be formulated for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Compositions for oral administration can be formulated according to any method known to those of skill in the art. Such compositions can contain one or more agents selected from sweetening agents, flavoring agents, coloring agents, antioxidants, and preserving agents in order to provide pharmaceutically elegant and palatable preparations.

Tablets generally contain the active ingredient in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release.

Compositions for oral administration can be formulated as hard gelatin capsules wherein the active ingredient is mixed with an inert solid diluent (such as calcium carbonate, calcium phosphate, or kaolin), or as soft gelatin capsules wherein the active ingredient is mixed with water or an oil medium (such as peanut oil, liquid paraffin, or olive oil).

### IV. Methods for Interferon Stimulation and Disease Treatment

Also provided herein are methods for stimulating interferon production in a cell. The methods include contacting the cell with an effective amount of a peptide construct as described above, a hexokinase inhibitor, a lactate dehydrogenase inhibitor, or a combination thereof. “Contacting” cells may include addition of a MAVS peptide construct to a cell culture in vitro, inducing expression of the MAVS peptide construct in cell culture using a nucleic acid or vector as described above, administering a MAVS peptide construct to a subject (e.g., in conjunction with a pharmaceutical composition as described above), or inducing expression of the MAVS peptide construct in the subject. Stimulating interferon production generally includes contacting the cells with an amount of the MAVS peptide construct, hexokinase inhibitor, or lactate dehydrogenase inhibitor sufficient to increase the level of interferon expression as compared to the level of interferon expressed in the absence of the MAVS construct, hexokinase inhibitor, or lactate dehydrogenase inhibitor. For example, contacting the cells with the MAVS peptide construct, hexokinase inhibitor, or lactate dehydrogenase inhibitor can result in increases ranging from about 1% to about 99% or higher, e.g., from about 100-200%, or from about 100-250%, or from about 100%-500%. In some embodiments, interferon expression may be increased by an order of magnitude or several orders of magnitude. Interferon expression levels may be assessed by a number of techniques, include via immunoassay or quantitative PCR as described in more detail below.

Also provided herein are methods for treating viral infections. The methods include administering to a subject in need thereof a therapeutically effective amount of a peptide construct as described above or a therapeutically effective amount of a pharmaceutical composition as described above. RIG-I is an RNA sensor that binds cytosolic RNAs prior to recruitment of MAVS. The sensing of RNA viruses is a major function of RIG-I and other RIG-I-like receptors (RLRs), and recognition of some DNA viruses by members of this protein family has also been demonstrated (Kell et al., 2015). Accordingly, some embodiments of the present disclosure provide methods for treating viral infections wherein the infection is caused by an RNA virus. Infections caused by negative-sense RNA viruses and positive-sense viruses can be treating using the methods provided herein. The infections may be caused by viruses including, but not limited to, paramyxoviridae (e.g., measles virus, Sendai virus, parainfluenzviruses, and the like), filoviridae (e.g., Ebola virus, Marburg virus, and the like), bornaviridae (e.g., Borna disease virus and the like), rhabdoviridae (e.g., vesicular stomatitis virus and the like), arteriviridae (e.g., porcine respiratory and reproductive syndrome virus (PRRSV) and like viruses that infect non-human primates), arenaviridae (e.g., Junin virus, Lassa virus, and the like), bunyaviridae (e.g., Hantaviruses, nairoviruses, and the like), orthomyxoviridae (e.g., influenza A, B, and C viruses, and the like), and flaviviridae (e.g., hepatitis C virus, Dengue virus, West Nile virus, Zika virus, and the like).

In some embodiments, the viral infection is a paramyxovirus infection, e.g., a respirovirus infection such as a Sendai virus (SEV) infection, a human parainfluenza virus 1 (HPIV-1) infection, or a human parainfluenza virus 3 (HPIV-3) infection. In some embodiments, the viral infection is a rhabdovirus infection such as a vesicular stomatitis virus (VSV) infection.

MAVS peptide constructs can be administered at any suitable dose in the methods provided herein. In general, a MAVS peptide construct is administered at a dose ranging from about 1 microgram to about 1000 milligrams per kilogram of a subject's body weight (i.e., about 0.001-1000 mg/kg). The dose of MAVS peptide construct can be, for example, about 0.001-1000 mg/kg, or about 0.01-500 mg/kg, or about 0.01-250 mg/kg, or about 0.01-100 mg/kg, or about 0.1-100 mg/kg, or about 0.1-50 mg/kg, or about 0.1-10 mg/kg. The dose of MAVS peptide construct can be about 0.001, 0.005, 0.01, 0.05, 0.1, 0.5, 1, 5, 25, 50, 75, 100, 200, 250, 300, 400, 500, 600, 700, 800, 900, or 1000 mg/kg. The dosages can be varied depending upon the requirements of the patient, the severity of the infection being treated, and the particular formulation being administered. The dose administered to a patient should be sufficient to result in a beneficial therapeutic response in the patient. The size of the dose will also be determined by the existence, nature, and extent of any adverse side-effects that accompany the administration of the drug in a particular patient. Determination of the proper dosage for a particular situation is within the skill of the typical practitioner. The total dosage can be divided and administered in portions over a period of time suitable to treat to the disease or condition.

MAVS peptide constructs can be administered for periods of time which will vary depending upon the nature of the infection, its severity, and the overall condition of the subject to whom the MAVS peptide construct is administered. Administration can be conducted, for example, hourly, every 2 hours, three hours, four hours, six hours, eight hours, or twice daily including every 12 hours, or any intervening interval thereof. Administration can be conducted once daily, or once every 36 hours or 48 hours, or once every month or several months. Following treatment, a subject can be monitored for changes in his or her condition and for alleviation of the symptoms of the disorder. The dosage of the MAVS peptide construct can either be increased in the event the subject does not respond significantly to a particular dosage level, or the dose can be decreased if an alleviation of the symptoms of the disorder is observed, or if the disorder has been remedied, or if unacceptable side effects are seen with a particular dosage. Administration of the MAVS peptide construct may be conducted over periods of time ranging from a few days to several weeks, months, or years.

Interferon stimulation may be enhanced in certain instances by using a MAVS peptide construct in combination with other agents that modulate levels of lactate production in target cells or tissues. Lactate itself is formed from pyruvate, the end-product of anaerobic glycolysis. As such, agents to be used in combination with the peptide constructs of the present disclosure may inhibit the production of lactate from pyruvate (e.g., by inhibition of lactate dehydrogenase), or such agents may inhibit one or more steps of the glycolytic pathway (e.g., by inhibition of hexokinase at the beginning of the glycolytic pathway). Examples of lactate dehydrogenase inhibitors include, but are not limited to, oxamic acid and salts thereof such as sodium oxamate; 1H-pyrazol-1-yl-thiazoles as described in WO 2018/005807; substituted benzo[d][1,3]dioxoles as described in US 2018/0015068; substituted N-hydroxyindoles as described by Granchi et al. (2011), and substituted dihydroxynaphthoic acids as described by Deck et al. (1998). Examples of hexokinase inhibitors include, but are not limited to, 2-deoxyglucose; 3-bromopyruvate; and other pyruvate analogs as described in U.S. Pat. No. 9,849,103. Hexokinase inhibitors and/or lactate dehydrogenase inhibitors may be administered to a subject prior to administration of a MAVS peptide construct, concomitantly with administration of a MAVS peptide construct, or after administration of a MAVS peptide construct. Hexokinase inhibitors and/or lactate dehydrogenase inhibitors may be co-formulated with MAVS peptide construct in pharmaceutical compositions such as those as described above.

Accordingly, some embodiments of the present disclosure provide methods for treating viral infections which further include administering to the subject a lactate dehydrogenase (LDH) inhibitor, a hexokinase (HK) inhibitor, an antiviral agent, or a combination thereof. In some embodiments, the LDH inhibitor is sodium oxamate. In some embodiments, the HK inhibitor is 2-deoxyglucose. Methods for treating viral infections may include administration of one or more antiviral agents including, but not limited, to an RNA polymerase inhibitor (e.g., ribavirin, taribavirin, favipiravir, or the like); a matrix 2 inhibitor (e.g., amantadine, rimantadine, or the like), a neuraminidase inhibitor (e.g., laninamivir, oseltamivir, peramivir, zanamivir, or the like); a protease inhibitor (e.g., HCV NS3 inhibitors such as asunaprevir, simeprevir, vaniprevir, or the like); an HCV NS5A inhibitor (e.g., daclatasvir, elbasvir, ledipasvir, ombitasvir, velpatasvir, or the like); an HCV NS5B inhibitor (e.g., beclabuvir, dasabuvir, sofosbuvir, or the like). Other anti-viral agents, including those described by De Clercq et al. (2016) may be useful depending on the nature of the particular infection being treated. One or more anti-viral agents may be administered to a subject prior to administration of a MAVS peptide construct, concomitantly with administration of a MAVS peptide construct, or after administration of a MAVS peptide construct. Anti-viral agents may be co-formulated with MAVS peptide construct in pharmaceutical compositions such as those as described above.

Also provided herein are methods for treating cancer. The methods include administering to a subject in need thereof a therapeutically effective amount of a peptide construct as described above or a therapeutically effective amount of a pharmaceutical composition as described above. Interferons produced by cancerous cells or tumor-infiltrating immune cells are now understood to contribute cancer immune-surveillance, as detailed by Zitvogel et al. (2015). For example, IFNs can promote maturation of dendritic cells and migration to lymph nodes, increase the cytotoxicity of natural killer cells and cytotoxic T lymphocytes, increase macrophage-associate inflammation, and decrease the immunosuppressive function of regulatory T cells. Stimulating IFN production using the methods provided herein can therefore improve the treatment of a variety of cancers including, but not limited to, chronic myeloid leukemia, hairy cell leukemia, melanoma, multiple myeloma, non-Hodgkin lymphoma, acute myeloid leukemia, prostate cancer (including castration-resistant prostate cancer), chronic lymphocytic leukemia, cutaneous lymphomas, polycythemia vera, relapsed follicular lymphoma, systemic mastocytosis, and testicular cancer (including testicular teratoma). Other cancers include fibrosarcoma, myxosarcoma, liposarcoma, chondrosarcoma, osteogenic sarcoma, chordoma, angiosarcoma, endotheliosarcoma, lymphangiosarcoma, lymphangioendothelio-sarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, small cell lung carcinoma, bladder carcinoma, lung cancer, epithelial carcinoma, glioma, glioblastoma multiform, astrocytoma, medulloblastoma, craniopharyngioma, ependymoma, pinealoma, hemangioblastoma, acoustic neuroma, oligodendroglioma, meningioma, skin cancer, neuroblastoma, or retinoblastoma.

Methods for treating cancer may include administration of one or more anti-cancer agents including, but are not limited to, a chemotherapeutic agent (e.g., carboplatin, paclitaxel, pemetrexed, or the like), a tyrosine kinase inhibitor (e.g., erlotinib, crizotinib, osimertinib, or the like), and an immunotherapeutic agent (e.g., pembrolizumab, nivolumab, durvalumab, atezolizumab, or the like). One or more anti-cancer agents may be administered to a subject prior to administration of a MAVS peptide construct, concomitantly with administration of a MAVS peptide construct, or after administration of a MAVS peptide construct. Anti-cancer agents may be co-formulated with MAVS peptide construct in pharmaceutical compositions such as those as described above. In some embodiments, methods for treating cancer includes the administration of radiotherapy, e.g., external beam radiation; intensity modulated radiation therapy (IMRT); brachytherapy (internal or implant radiation therapy); stereotactic body radiation therapy (SBRT)/stereotactic ablative radiotherapy (SABR); stereotactic radiosurgery (SRS); or a combination of such techniques. In some embodiments, methods for treating cancer further include administration of a lactate dehydrogenase (LDH) inhibitor, a hexokinase (HK) inhibitor, or a combination thereof. In some embodiments, the LDH inhibitor is sodium oxamate. In some embodiments, the HK inhibitor is 2-deoxyglucose. Dosing and administration of the MAVS peptide construct may be varied as set forth above with respect to the treatment of viral infections.

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a peptide construct comprising a mitochondrial antiviral-signaling protein (MAVS) peptide and a cell penetration peptide.

Embodiment 2 is the peptide construct of embodiment 1, wherein the MAVS peptide comprises a MAVS transmembrane domain.

Embodiment 3 is the peptide construct of embodiment 2, wherein the MAVS transmembrane domain comprises an amino acid sequence having at least 70% identity to SEQ ID NO:2.

Embodiment 4 is the peptide construct of any one of embodiment(s) 1-3, wherein the cell penetration peptide is an HIV-1 Tat peptide.

Embodiment 5 is the peptide construct of embodiment 4, wherein the HIV-1 Tat peptide comprises an amino acid sequence having at least 70% identity to SEQ ID NO:6.

Embodiment 6 is the peptide construct of embodiment 5, wherein the cell penetration peptide comprises the amino acid sequence of SEQ ID NO:6 and the MAVS peptide comprises an amino acid sequence having at least 70% identity to SEQ ID NO:2.

Embodiment 7 is the peptide construct of any one of embodiment(s) 1-5, wherein the C-terminus of the cell penetration peptide is linked to the N-terminus of the MAVS peptide.

Embodiment 8 is the peptide construct of embodiment 1, comprising the amino acid sequence of SEQ ID NO:24.

Embodiment 9 is a nucleic acid encoding a peptide construct according to any one of embodiment(s) 1-8.

Embodiment 10 is a vector comprising the nucleic acid of embodiment 9.

Embodiment 11 is a host cell comprising the nucleic acid of embodiment 9 or the vector of embodiment(s) 10.

Embodiment 12 is a pharmaceutical composition comprising a peptide construct according to any one of embodiment(s) 1-8 and a pharmaceutically acceptable excipient.

Embodiment 13 is a method of stimulating interferon production in a cell, the method comprising contacting the cell with an effective amount of a peptide construct according to any one of embodiment(s) 1-8, a hexokinase inhibitor, a lactate dehydrogenase inhibitor, or a combination thereof.

Embodiment 14 is a method of treating a viral infection, the method comprising administering to a subject in need thereof a therapeutically effective amount of a peptide construct according to any one of embodiment(s) 1-8 or a therapeutically effective amount of a pharmaceutical composition according to claim 12.

Embodiment 15 is the method of embodiment 14, wherein the viral infection is caused by an RNA virus.

Embodiment 16 is the method of embodiment 14, wherein the viral infection is a parainfluenza virus infection.

Embodiment 17 is the method of any one of embodiment(s) 14-16, further comprising administering to the subject a lactate dehydrogenase (LDH) inhibitor, a hexokinase (HK) inhibitor, an antiviral agent, or a combination thereof.

Embodiment 18 is the method of embodiment 17, wherein the LDH inhibitor is sodium oxamate.

Embodiment 19 is the method of embodiment 17, wherein the HK inhibitor is 2-deoxyglucose.

Embodiment 20 is a method of treating cancer, the method comprising administering to a subject in need thereof a therapeutically effective amount of a peptide construct according to any one of embodiment(s)s 1-8 or a therapeutically effective amount of a pharmaceutical composition according to claim 12.

Embodiment 21 is the method of embodiment 20, further comprising administering an anti-cancer agent to the subject.

Embodiment 22 is the method of embodiment 20, further comprising administering radiation therapy to the subject.

Embodiment 23 is the method of any one of embodiment(s) 20-22, further comprising administering to the subject a lactate dehydrogenase (LDH) inhibitor, a hexokinase (HK) inhibitor, or a combination thereof.

Embodiment 24 is the method of embodiment 23, wherein the LDH inhibitor is sodium oxamate.

Embodiment 25 is the method of embodiment 23, wherein the HK inhibitor is 2-deoxyglucose.

References referred to in this disclosure include:
1. Baker, J. C., et al. (2000). Marked differences between two isoforms of human pyruvate dehydrogenase kinase. J Biol Chem 275, 15773-15781.
2. Brand, A., et al. (2016). LDHA-Associated Lactic Acid Production Blunts Tumor Immunosurveillance by T and NK Cells. Cell Metab 24, 657-671.
3. Burdette, D. L., et al. (2011). STING is a direct innate immune sensor of cyclic di-GMP. Nature 478, 515-518.
4. Chang, C. H., et al. (2013). Posttranscriptional control of T cell effector function by aerobic glycolysis. Cell 153, 1239-1251.
5. Chen, W., et al. (2013a). Induction of Siglec-G by RNA viruses inhibits the innate immune response by promoting RIG-I degradation. Cell 152, 467-478.
6. Chen, Y., et al. (2013b). Human infections with the emerging avian influenza A H7N9 virus from wet market poultry: clinical analysis and characterisation of viral genome. Lancet 381, 1916-1925.
7. Colegio, et al. (2014). Functional polarization of tumour-associated macrophages by tumour-derived lactic acid. Nature 513, 559-563.
8. Crane, et al. (2014). Immune evasion mediated by tumor-derived lactate dehydrogenase induction of NKG2D ligands on myeloid cells in glioblastoma patients. Proc Natl Acad Sci USA 111, 12823-12828.
9. Davies, L. C., et al. (2017). Peritoneal tissue-resident macrophages are metabolically poised to engage microbes using tissue-niche fuels. Nat Commun 8, 2074.
10. DeBerardinis, R. J., et al. (2012). Cellular metabolism and disease: what do metabolic outliers teach us? Cell 148, 1132-1144.
11. Deck, L. M., et al. (1998). J. Med. Chem., 41 (20), 3879-3887.
12. De Clercq, E., et al. (2016). Approved Antiviral Drugs over the Past 50 Years. Clin Microbiol Rev 29:695-747.
13. DeWaal, D., et al. (2018). Hexokinase-2 depletion inhibits glycolysis and induces oxidative phosphorylation in hepatocellular carcinoma and sensitizes to metformin. Nat Commun 9, 446.
14. Faubert, B., et al. (2017). Lactate Metabolism in Human Lung Tumors. Cell 171, 358-371 e359.
15. Feng, H., et al. (2017). NLRX1 promotes immediate IRF1-directed antiviral responses by limiting dsRNA-activated translational inhibition mediated by PKR. Nat Immunol 18, 1299-1309.
16. Gack, M. U., et al. (2007). TRIM25 RING-finger E3 ubiquitin ligase is essential for RIG-I-mediated antiviral activity. Nature 446, 916-920.
17. Ganeshan, K., et al. (2014). Metabolic regulation of immune responses. Annu Rev Immunol 32, 609-634.
18. Granchi, et al. Guan, K., et al. (2013). MAVS regulates apoptotic cell death by decreasing K48-linked ubiquitination of voltage-dependent anion channel 1. Mol Cell Biol 33, 3137-3149.
19. Granchi, C., et al. (2011) J. Med. Chem., 54 (6), 1599-1612
20. Guidotti, G., et al. (2017) Trends in Pharmacological Sciences, 38 (4), 406-424
21. Honda, K., et al. (2006). Type I interferon [corrected] gene induction by the interferon regulatory factor family of transcription factors. Immunity 25, 349-360.
22. Hou, F., et al. (2011). MAVS forms functional prion-like aggregates to activate and propagate antiviral innate immune response. Cell 146, 448-461.
23. Hunt, L., et al. (2015). Clinical presentation, biochemical, and haematological parameters and their association with outcome in patients with Ebola virus disease: an observational cohort study. Lancet Infect Dis 15, 1292-1299.
24. Ivashkiv, et al. (2014). Regulation of type I interferon responses. Nat Rev Immunol 14, 36-49.
25. Jin, S., et al. (2017). Tetherin Suppresses Type I Interferon Signaling by Targeting MAVS for NDP52-Mediated Selective Autophagic Degradation in Human Cells. Mol Cell 68, 308-322 e304.
26. Kawai, T., et al. (2010). The role of pattern-recognition receptors in innate immunity: update on Toll-like receptors. Nat Immunol 11, 373-384.
27. Kell, A. M., et al. (2015). Virology 0, 110-121.
28. Liu, X., et al. (2018). Acetate Production from Glucose and Coupling to Mitochondrial Metabolism in Mammals. Cell 175, 502-513 e513.
29. Lunt, S. Y., et al. (2011). Aerobic glycolysis: meeting the metabolic requirements of cell proliferation. Annu Rev Cell Dev Biol 27, 441-464.
30. MacMicking, J. D. (2012). Interferon-inducible effector mechanisms in cell-autonomous immunity. Nat Rev Immunol 12, 367-382.
31. Michelakis, E. D., et al. (2008). Dichloroacetate (DCA) as a potential metabolic-targeting therapy for cancer. Br J Cancer 99, 989-994.
32. Milletti, F. (2012). Cell-penetrating peptides: classes, origin, and current landscape. Drug Discov Today 17, 850-860.
33. Nakazawa, M. S., et al. (2016). Oxygen availability and metabolic adaptations. Nat Rev Cancer 16, 663-673.
34. Parks, S. K., et al. (2013). Disrupting proton dynamics and energy metabolism for cancer therapy. Nat Rev Cancer 13, 611-623.
35. Patra, K. C., et al. (2013). Hexokinase 2 is required for tumor initiation and maintenance and its systemic deletion is therapeutic in mouse models of cancer. Cancer Cell 24, 213-228.
36. Pavlova, N. N., et al. (2016). The Emerging Hallmarks of Cancer Metabolism. Cell Metab 23, 27-47.
37. Peng, M., et al. (2016). Aerobic glycolysis promotes T helper 1 cell differentiation through an epigenetic mechanism. Science 354, 481-484.
38. Prezma, T., et al. (2013). VDAC1-based peptides: novel pro-apoptotic agents and potential therapeutics for B-cell chronic lymphocytic leukemia. Cell Death Dis 4, e809.
39. Roberts, D. J., et al. (2015). Hexokinase II integrates energy metabolism and cellular protection: Akting on mitochondria and TORCing to autophagy. Cell Death Differ 22, 364.
40. Rossignol, R., et al. (2004). Energy substrate modulates mitochondrial structure and oxidative capacity in cancer cells. Cancer Res 64, 985-993.
41. Schlee, M., et al. (2016). Discriminating self from non-self in nucleic acid sensing. Nat Rev Immunol 16, 566-580.
42. Schoggins, J. W., et al. (2011). A diverse range of gene products are effectors of the type I interferon antiviral response. Nature 472, 481-485.
43. Seth, R. B., et al. (2005). Identification and characterization of MAVS, a mitochondrial antiviral signaling protein that activates NF-kappaB and IRF 3. Cell 122, 669-682.
44. Stark, G. R., et al. (2012). The JAK-STAT pathway at twenty. Immunity 36, 503-514.
45. Tamura, T., et al. (2008). The IRF family transcription factors in immunity and oncogenesis. Annu Rev Immunol 26, 535-584.
46. Trinchieri, G. (2010). Type I interferon: friend or foe? J Exp Med 207, 2053-2063.
47. Weinberg, F., et al. (2010). Mitochondrial metabolism and ROS generation are essential for Kras-mediated tumorigenicity. Proc Natl Acad Sci USA 107, 8788-8793.
48. Wolf, A. J., et al. (2016). Hexokinase Is an Innate Immune Receptor for the Detection of Bacterial Peptidoglycan. Cell 166, 624-636.
49. Xie, H., et al. (2014). Targeting lactate dehydrogenase—a inhibits tumorigenesis and tumor progression in mouse models of lung cancer and impacts tumor-initiating cells. Cell Metab 19, 795-809.
50. You, F., et al. (2013). ELF4 is critical for induction of type I interferon and the host antiviral response. Nat Immunol 14, 1237-1246.
51. Zeng, W., et al. (2009). Key role of Ubc5 and lysine-63 polyubiquitination in viral activation of IRF3. Mol Cell 36, 315-325.
52. Zhang, J., et al. (2017). c-Src phosphorylation and activation of hexokinase promotes tumorigenesis and metastasis. Nat Commun 8, 13732.
53. Zhao, W., et al. (2014). Steroid receptor coactivator-3 regulates glucose metabolism in bladder cancer cells through coactivation of hypoxia inducible factor 1alpha. J Biol Chem 289, 11219-11229.
54. Zhao, Y., et al. (2016). In vivo monitoring of cellular energy metabolism using SoNar, a highly responsive sensor for NAD(+)/NADH redox state. Nat Protoc 11, 1345-1359.
55. Zhong, X., et al. (2017). CUE domain-containing protein 2 promotes the Warburg effect and tumorigenesis. EMBO Rep 18, 809-825.
56. Zhu, J., et al. (2014). Antiviral activity of human OASL protein is mediated by enhancing signaling of the RIG-I RNA sensor. Immunity 40, 936-948.
57. Zitvogel, L., et al. (2015). Type I interferons in anticancer immunity. Nat Rev Immunol 15, 405-414.

### EXAMPLES

### Example 1. Materials and Methods

Mice. Ldha^{+/+} and Ldha^{-/-} mice on a C57BL/6J background were previously described (Xie et al., 2014). All animal procedures were approved by Institutional Animal Care and Use Committee in Wake Forest University School of Medicine. Littermates of the same sex were randomly assigned to experimental groups. 6-8 weeks old mice were treated with tamoxifen (20 mg/ml in corn oil) or coin oil by intraperitoneal injection. According to known protocols for inducible Cre driver lines, LDHA exon2 deletion was confirmed by genotyping 10 days after final injection. Mice were then used for peritonea macrophage isolation or virus infection by intraperitoneal injection. IFN-β concentration in supernatants of peritoneal macrophage or sera from Ldha^{+/+} and Ldha^{-/-} mice were determined by using the mouse IFN-β ELISA kit (Thermo Fisher 424001) according to the manufacturer's protocol. All the analyses were performed blindly. Ldha^{-/-} mice were randomly allocated into experimental groups for further treatment and cell samples were allocated based on the genotype of interest.

Cells. HEK293, Hep3B, Raw264.7 cells and immortalized bone marrow macrophage (iBMM) cells were cultured in DMEM medium supplied with 10% FBS, 2 mM glutamine, penicillin (100 U/mL) and streptomycin (100 mg/mL). THP-1 cells were cultured in RPMI-1640 medium supplied with 10% FBS, 2 mM glutamine, penicillin (100 U/mL) and streptomycin (100 mg/mL). Primary peritoneal macrophages were isolated from mice 14 days after tamoxifen intraperitoneal injection as previously described (Chen et al., 2013a). Cells were plated 24 hours before transfection with Poly(I:C) (1 μg/ml), HTDNA (1 μg/ml) or siRNA (10 nM) by Lipofectamine 2000 (Thermo Fisher Scientific). THP-1 cells were pretreated by PMA (100 ng/mL) overnight for differentiation before the next manipulation. To generate shRNA knockdown cells, HEK293T cells were prepared and co-transfected with either luciferase (shLuc) or target gene shRNA with packaging plasmid (pHelper) and envelop plasmid (pEnv) by using the calcium phosphate transfection method. Medium was changed 6 hours later and virus particles were harvested after another 48 hours to infect parental cells, then selected by puromycin.

Viruses. VSV and HSV-1 were propagated and tittered by plague assay on Vero cells. For in vivo cytokine production studies, age and sex-matched groups of littermate mice were intraperitoneally injected with VSV or HSV-1 (2×10⁷ pfu/g). For cell based assays, cells were infected with VSV-GFP (0.1 MOI) or SeV (1 MOI) for the indicated periods of time and cytokine production was analyzed by Q-PCR or ELISA.

Immunoblotting (IB), Immunoprecipitation (IP) and Pull down assays. For immunoblotting, whole cell lysates were prepared in E1A buffer (50 mM Hepes, pH 7.6, 250 mM NaCl, 0.1% Nonidet P-40, 5 mM EDTA) supplemented with complete protease inhibitor cocktail (Roche, 04693132001). Cell lysates were separated by SDS-PAGE and proteins were visualized by enhanced chemiluminescence according to the manufacturer's instructions (ThermoFisher Scientific). For protein-protein interactions, cells were lysed by RIPA lysis buffer (150 mM NaCl, 0.5% NP-40, 5 mM EDTA) containing a mixture of protease inhibitor cocktail (Roche). Primary antibodies were incubated with protein agarose A/G beads for 30 min at room temperature, followed by incubating with cell lysates for 3 hours with rotating at RT. The beads were washed four times with lysis buffer and analyzed by immunoblotting. For Biotin-lactate pull down assays, Dynabeads™ MyOne™ Streptavidin T1 (ThermoFisher) were preincubated with free biotin or biotin-labeled lactate in PBS for 1 hour at RT, and then incubated with cell lysates overnight with rotating at 4° C. The beads were washed 3-4 times and analyzed by immunoblotting.

Lactate treatment. For lactate addiction assay, two forms of lactate were used: the acid form of lactate (L-lactic acid), which was used for in vitro cell based study and in vitro MAVS aggregation assay; and the basic form of lactate (sodium lactate), which was used for in vivo animal experiments.

Immunofluorescent Confocal Microscopy. Cells were fixed with 3% paraformaldehyde for 20 min at 25° C. and permeabilized for 20 min with 0.5% Tween-20. Samples were blocked with 2% goat serum in phosphate-buffered saline (PBS) for 30 min. Anti-MAVS (1:500, Santa Cruz) and anti-TAT (1:500, Abcam) antibodies were used to detect the MAVS protein and TM peptides, respectively. Staining was visualized with secondary antibodies and the images were captured with a digital camera under a confocal microscope.

RNA extraction and quantitative RT-PCR. Total RNA was extracted via TRIzol reagent (Ambion 15596-018). Total RNAs (0.5-1 μg) were subjected to reverse transcription with PrimeScript RT Master Mix (Takara, DRR036A). To determine relative mRNA level, Q-PCR was performed using universal SYBR Green Supermix (Bio-Rad 172-5125) and gene expression was normalized to that of GAPDH or HPRT. Primers used for Q-PCR were are listed in Table 2.

TABLE 2
PCR Primers

| | SEQ | |
|---|---|---|
| Primer | ID NO: | Sequence |
| Human IFNb- | 25 | TCCAAATTGCTCTCCTGTTG |
| qPCR-F | | |
| Human IFNb- | 26 | GCAGTATTCAAGCCTCCCAT |
| qPCR-R | | |
| Mouse IFNb- | 27 | TCCGAGCAGAGATCTTCAGGAA |
| qPCR-F | | |
| Mouse IFNb- | 28 | TGCAACCACCACTCATTCTGAG |
| qPCR-R | | |
| Human IL- | 29 | TGCAGAAAAAGGCAAAGAATCTAG |
| 6-qPCR-F | | |
| Human IL-6- | 30 | CGTCAGCAGGCTGGCATTT |
| qPCR-R | | |
| Mouse IL- | 31 | AGTTGCCTTCTTGGGACTGATG |
| 6-qPCR-F | | |
| Mouse IL- | 32 | GGGAGTGGTATCCTCTGTGAAGTCT |
| 6-qPCR-R | | |
| Mouse IFNa- | 33 | CCTGAACATCTTCACATCAAAGGA |
| qPCR-F | | |
| Mouse IFNa- | 34 | AGCTGCTGGTGGAGGTCATT |
| qPCR-R | | |
| Human GAPDH- | 35 | GAGTCAACGGATTTGGTCGT |
| qPCR-F | | |
| Human GAPDH- | 36 | TTGATTTTGGAGGGATCTCG |
| qPCR-R | | |
| Mouse HPRT- | 37 | CAGTCCCAGCGTCGTGATTAG |
| qPCR-F | | |
| Mouse HPRT- | 38 | AAACACTTTTTCCAAATCCTCGG |
| qPCR-R | | |
| Sev-qPCR-F | 39 | GCTGCCGACAAGGTGAGAGC |
| Sev-qPCR-R | 40 | GCCCGCCATGCCTCTCTCTA |
| VSV-qPCR-F | 41 | ACGGCGTACTTCCAGATGG |
| VSV-qPCR-R | 42 | CTCGGTTCAAGATCCAGGT |

Measurement of hexokinase activity and lactate level. For hexokinase activity detection, mitochondria were isolated from cells and pellet was lysed and subjected to Hexokinase activity measurement by using Hexokinase Colorimetric assay kit (Biovision K789-100). Secreted lactate level was measured by lactate Plus Test Strips (Nova Biomedical/fisher), whereas intracellular lactate level was measured by using lactate Colorimetric/Fluorometric assay kit (Biovision K607-100) according to manufacturer's protocol. For Seahorse analysis, cells with control or MAVS knockdown were prepared, the XF24 Extracellular Flux analyzer (Seahorse Biosciences, Billerica, Mass.) was used to measure extracellular acidification rate (ECAR).

In vivo and in vitro MAVS aggregation assays. In vivo MAVS aggregation was performed according to published protocol (Hou et al., 2011). In brief, mitochondria were isolated by using the Mitochondria isolation kit (Thermo 89874), and mitochondria pellet was suspended in 1×sample buffer (0.5×TBE, 10% glycerol, 2% SDS, and 0.0025% bromophenol blue) and subjected to Semi-denaturing detergent agarose gel electrophoresis (SDD-AGE). Samples were loaded onto a vertical 1.5% agarose gel (Bio-Rad). After electrophoresis in the running buffer (1×TBE and 0.1% SDS) for 40 min with a constant voltage of 80-100 V at 4° C., the proteins were transferred to Immobilon membrane (Millipore) for immunoblotting.

For in vitro MAVS aggregation, crude mitochondria were isolated and RIG-I activation was detected as previously described (Zeng et al., 2009). Briefly, each mixture contained 100 ng GST-RIG-I(N) and 50-100 ng ubiquitin chains (K63-Ub4 from Boston Biochem UC-310B) in buffer containing 20 mM HEPES-KOH (pH 7.0) and 10% (v/v) glycerol. After incubation at RT for 10 min in total 10 μl reaction system, 1 μl of reaction mixture was mixed with 10 μg of mitochondrial fraction in 10 μl Buffer B (20 mM HEPES-KOH [pH 7.0], 5 mM MgCl₂, and 0.25 M D-mannitol) at 30° C. for 30 min. Mitochondria fraction was then pelleted at 10,000 g for 10 min and washed twice with Buffer C (20 mM HEPES-KOH at pH 7.4, 0.5 mM EGTA, 0.25 MD-mannitol, and EDTA-free protease inhibitor cocktail) and then subjected to SDD-AGE.

Statistics. Statistical significance was identified by Student's t test. p values of less than 0.05 were considered statistically significant; *p<0.05, **p<0.01.

### Example 2. Downregulation of Glucose Metabolism Promotes RLR Induced Type-I IFN Production

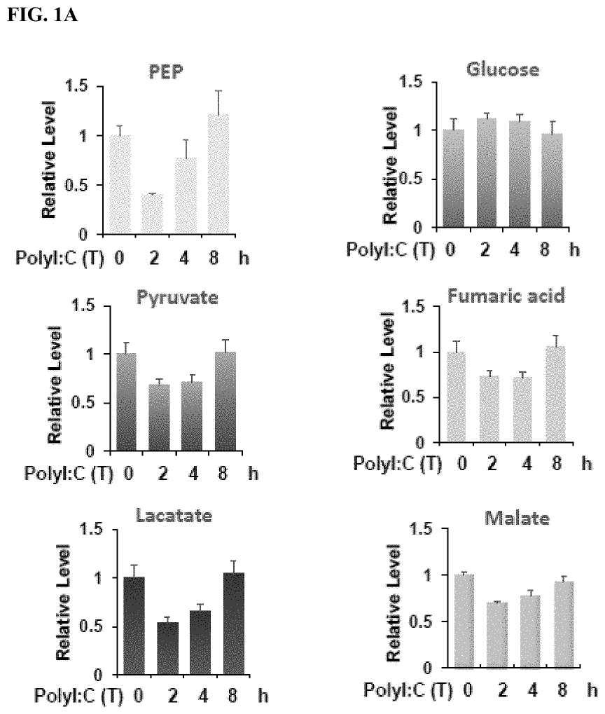

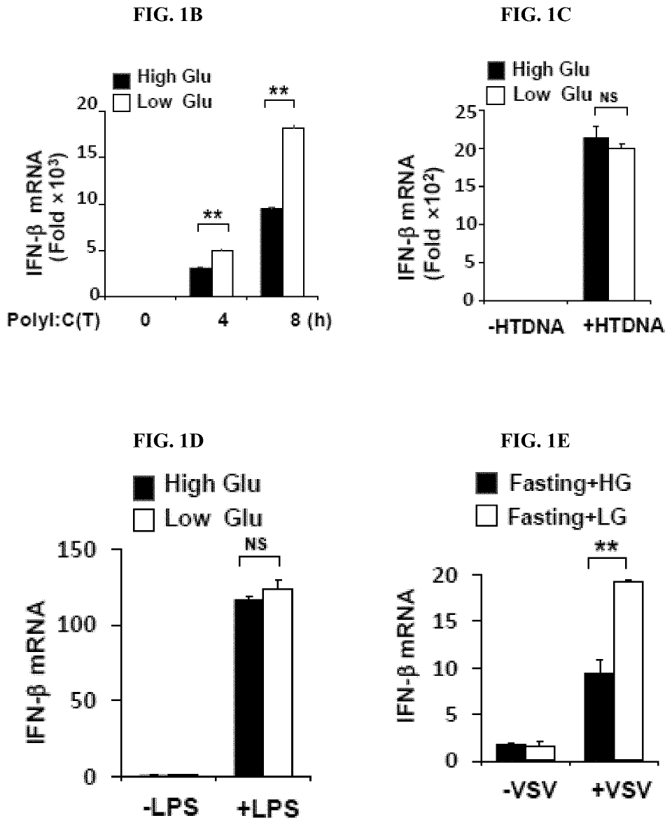

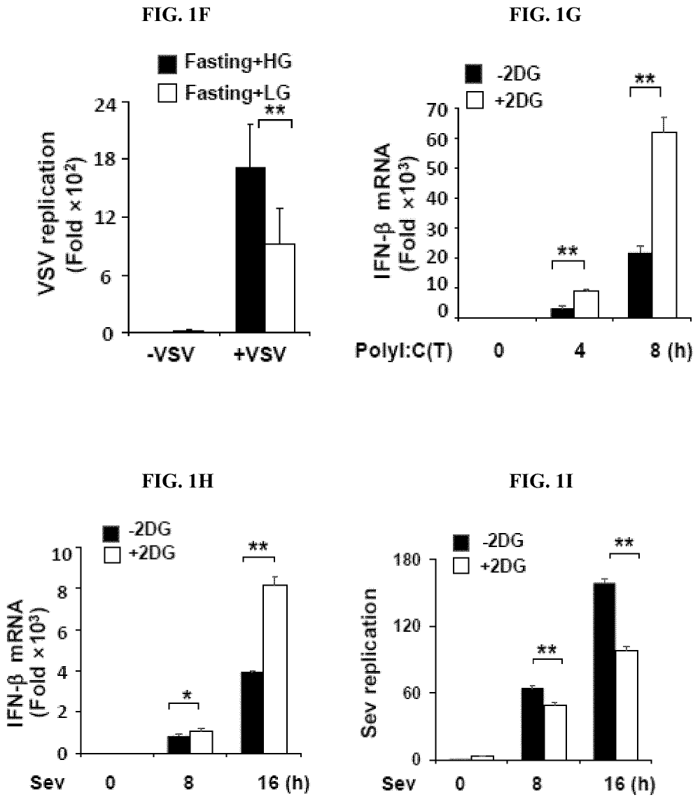

To investigate whether energy metabolism participates in regulating type-I IFN production, an unbiased, a systemic metabolomics approach was employed to examine the global metabolic changes during RLR-induced type-I IFN production. Changes of metabolites in glycolysis or oxidative phosphorylation were observed upon transfection of HEK293 cells with Poly(I:C) and harvesting for metabolomics analysis. The results of this analysis are shown in Table 3 and FIG. 1A. Intriguingly, most metabolic intermediates downstream of glucose, such as phosphoenolpyruvate (PEP), pyruvate and lactate, exhibited decreased levels at the initial stage of type-I IFN production. The levels of TCA intermediates, such as aconitate, succinate, fumaric acid and malate, were also significantly downregulated, likely resulting from the reduced level of pyruvate, which shunts into the TCA cycle for the production of these intermediates. However, oxaloacetate (OAA) level was not significantly affected (Table 3). As the direct pyruvate downstream metabolite, Acetyl-CoA production would be expected to decrease due to the reduction in pyruvate level, leading to the slower consumption of OAA for citrate production and thus maintaining the OAA level. These results suggest that glucose metabolism is impaired during the RLR signaling activation.

TABLE 3
Metabolomics Analysis

| | Hours Post PolyI:C Transfection | | | |
|---|---|---|---|---|
| | (Relative Quantity (polyI:C)) | | | |
| Metabolic Intermediates | 0 | 2 | 4 | 8 |
| Glucose (179.0/89.0) | 1.000017 | 1.118381 | 1.093305 | 0.964092 |

Glycolysis

| | | | | |
|---|---|---|---|---|
| G1P/G6P/F6P/F1P | 1.00015 | 0.712041 | 0.870649 | 1.033896 |
| (259.0/97.0) | | | | |
| F16BP/F26BP/G16BP | 0.999977 | 0.566603 | 0.910306 | 1.127 |
| (339.0/97.0) | | | | |
| D-GA3P (168.9/97.0) | 1.000497 | 0.504185 | 0.627774 | 0.998 |
| DHAP (168.9/78.8) | 1.001771 | 0.633399 | 0.934983 | 1.100532 |
| 2/3-Phosphoglyceric | 1.000468 | 0.499355 | 0.798496 | 1.118427 |
| Acid (185.0/97.0) | | | | |
| PEP (166.9/79.0) | 1.000638 | 0.396797 | 0.77056 | 1.061993 |
| Pyruvate (87.0/43.0) | 1.000 | 0.683 | 0.711547 | 1.022948 |
| Lactate (89.0/43.0) | 0.999953 | 0.536 | 0.657 | 1.049574 |

TCA Cycle

| | | | | |
|---|---|---|---|---|
| 3HBA (103.0/59.0) | 0.99922 | 0.680825 | 0.77346 | 1.035117 |
| Aconitate (173.0/85.0) | 1.000003 | 0.765466 | 0.657623 | 0.77238 |
| Alpha-Ketoglutaric | 1.000059 | 0.940031 | 0.895868 | 0.965025 |
| Acid (145.0/101.0) | | | | |
| Succinate (117.0/73.0) | 0.999869 | 0.761605 | 0.879309 | 0.888622 |
| Fumaric Acid | 0.997643 | 0.727891 | 0.709425 | 1.056723 |
| (115.0/71.0) | | | | |
| Malate (133.0/115.0) | 0.999982 | 0.697897 | 0.784254 | 0.935265 |
| Maleic Acid | 0.999702 | 0.619412 | 0.756615 | 0.935247 |
| (115.0/71.0 (2)) | | | | |
| Malonic Acid | 1.02994 | 0.564421 | 0.694552 | 0.982343 |
| (103.0/41.0) | | | | |
| Oxalacetate | 0.999997 | 0.976986 | 1.006089 | 1.032176 |
| (131.0/113.0) | | | | |

Systemic glucose metabolism changes during RLR-induced type-I IFN production raised the possibility that glucose catabolism is involved in the regulation of type-I IFN production. To test this possibility, cells were incubated with high or low glucose to monitor RLR induced cytokine production and found low glucose-treated cells showed much stronger induction of IFN-β and IL-6 mRNA upon poly(I:C) transfection, but not HTDNA or LPS treatment, which activates stimulator of interferon genes (STING) and Toll-like receptor (TLR) signaling, respectively, compared with those under high glucose treatment (FIG. 1B-1D), indicative of the specificity of glucose change in regulating RLR signaling. Importantly, higher IFN-β induction and lower virus replication were observed in fasted mice treated with low glucose compared to those treated with high glucose (FIGS. 1E and 1F). Similarly, 2-deoxy-glucose (2-DG), which blocks glucose metabolism by inhibiting hexokinase (HK) activity, markedly enhanced IFN-β and IL-6 production driven by different RLR stimuli including poly(I:C) transfection, Sendai virus (SEV) and Vesicular stomatitis virus (VSV) infection, leading to the reduction in virus replication (FIG. 1G-1I). These data suggest that downregulation of glucose metabolism promotes RLR induced type-I IFN production.

### Example 3. Mitochondria Hexokinase Activity is Maintained by MAVS and Inactivated During RLR Activation

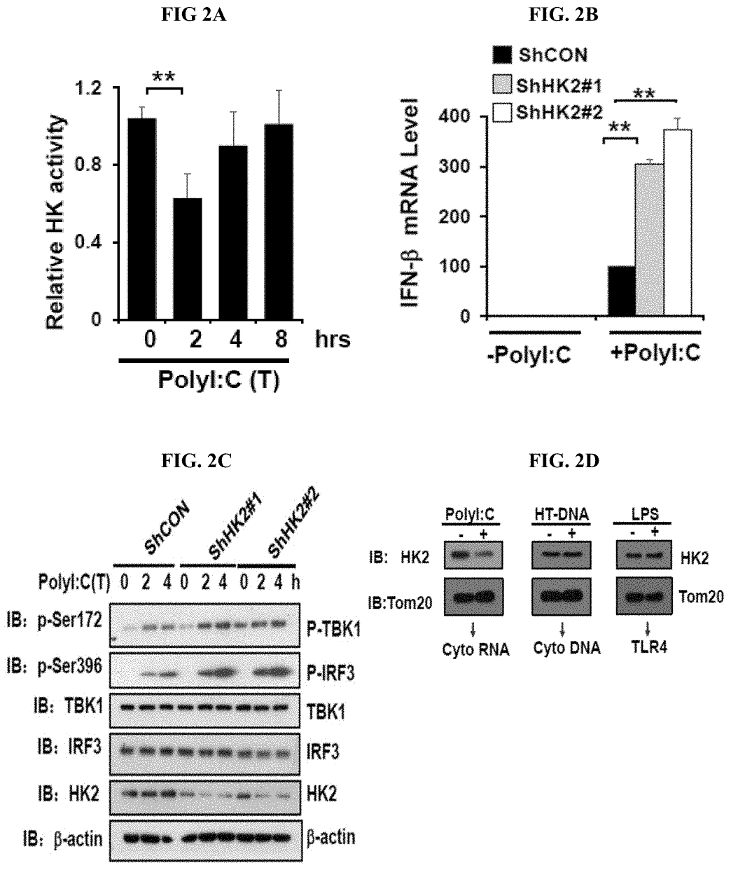

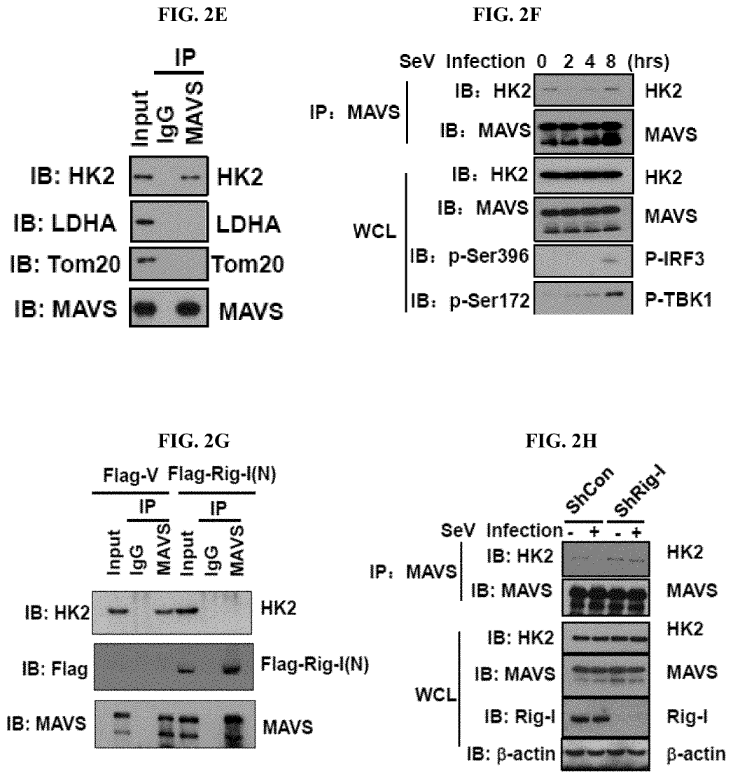

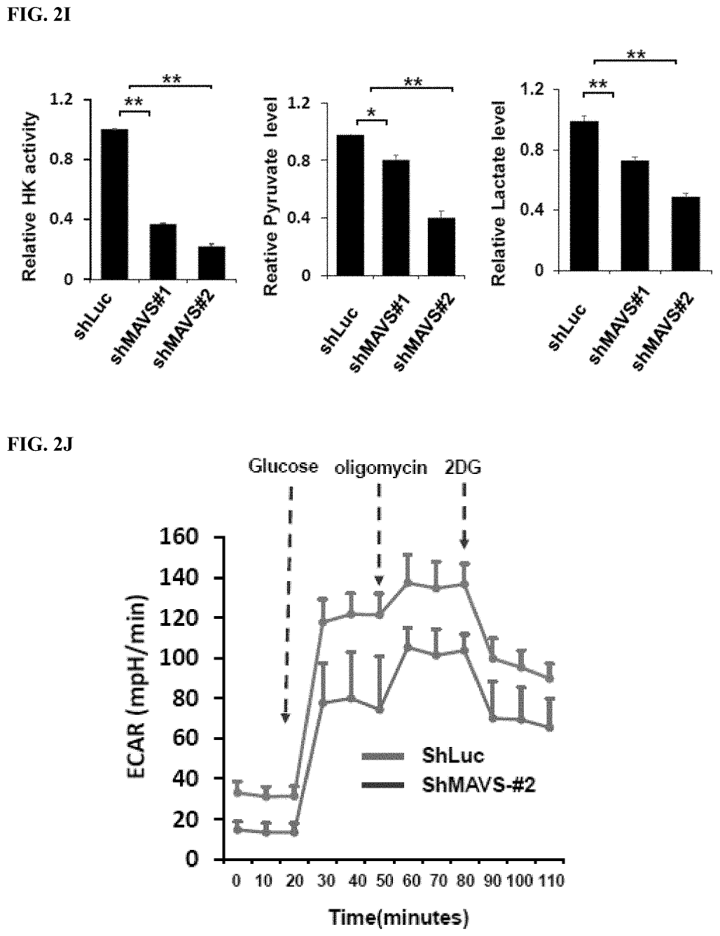

Since most glucose intermediates downstream of hexokinase (HK) were down-regulated during RLR-mediated type-I IFN induction, it is conceivable that HK activity might be affected during RLR activation. Indeed, HK activity decreased at early phase of RLR activation, correlated well with dynamic changes of glucose metabolites (FIG. 2A), indicating that a transient suppression of glucose metabolism through HK inhibition may be required for RLR-mediated type-I IFN induction. To further validate this idea, HK2, the major isoform of HK in most human cell types, was knocked down to mimic the suppression of glucose metabolism. Consistently, HK2 knockdown not only decreased glycolysis determined by reduced pyruvate and lactate levels, but also markedly enhanced TBK1-IRF3 signaling, cytokine production and SEV replication suppression upon RLR activation (FIGS. 2B-2C). Moreover, restoration of HK2 expression compromised the heightened effect of HK2 knockdown on IFN-β induction (data not shown).

The mitochondria localization of HK2 is required for its activation and full glycolytic function in cell (DeWaal et al., 2018; Roberts and Miyamoto, 2015; Wolf et al., 2016). To gain insight into how HK activity was downregulated during the early stages of RLR activation, the dependence of mitochondria HK2 levels upon RLR activation was examined. Of note, mitochondria HK2 decreased upon the Poly(I:C) transfection, but not HTDNA or LPS treatment (FIG. 2D), which might account for the downregulation of HK activity during RLR activation. Next, the mechanism underlying HK2 dissociation from mitochondria was explored. Interestingly, MAVS, as a mitochondria localized protein, physiologically interacted with HK2, but not with other mitochondria proteins, like Tom20 (FIG. 2E). Remarkably, the interaction between MAVS and HK2 declined during initial activation of RLR signaling triggered by Sev infection, but not STING signaling activation upon HTDNA transfection, correlated with the dissociation of HK2 from mitochondria (FIG. 2F). Moreover, a direct switch of MAVS binding from HK2 to RIG-I was observed in cells with ectopic expression of activated form of RIG-I (FIG. 2G). Furthermore, the dissociation of HK2 from MAVS upon RLR activation was not observed in RIG-I knockdown cells (FIG. 2H). These results suggested that RLR-triggered MAVS-RIG-I recognition hijacks HK2 binding to MAVS, leading to the impairment of HK mitochondria localization and activation.

As the downregulation of HK activity during RLR activation coincidences with RIG-I-MAVS recognition and subsequent MAVS activation upon RLR agonist stimulation (Schlee and Hartmann, 2016; Seth et al., 2005), the ability of MAVS to regulate mitochondria HK activity was studied. Interestingly, significant reduction of HK activity was observed in MAVS knockdown and knockout cells compared to control cells, accompanied by decreased pyruvate and lactate levels as well as extracellular acidification rate (ECAR) (FIG. 2I-2J). These results suggest that MAVS maintains HK activity.

HK associates with the mitochondrial outer membrane through its interaction with the voltage-dependent anion channel (VDAC), which is involved in glycolysis regulation (Roberts and Miyamoto, 2015; Wolf et al., 2016). To examine whether VDAC binding to HK2 is necessary for the interaction between HK2 and MAVS, a peptide that is known to disrupt HKs from VDAC (Prezma et al., 2013) was employed. Notably, disrupting VDAC1 and HK2 interaction by this peptide impaired the interaction of MAVS with HK2, indicating that binding to VDAC is required for HK2 interaction with MAVS (data not shown). Collectively, it can be concluded that MAVS is a previously unrecognized key regulator for HK, and switching MAVS binding from HK2 to RIG-I during RLR activation triggers HK2 release from mitochondria leading to impairing HK activity and subsequent glucose metabolism.

### Example 4. Anaerobic Glycolysis Inhibits RLR Triggered MAVS-TBK1-IRF3 Activation and Type-I IFN Production

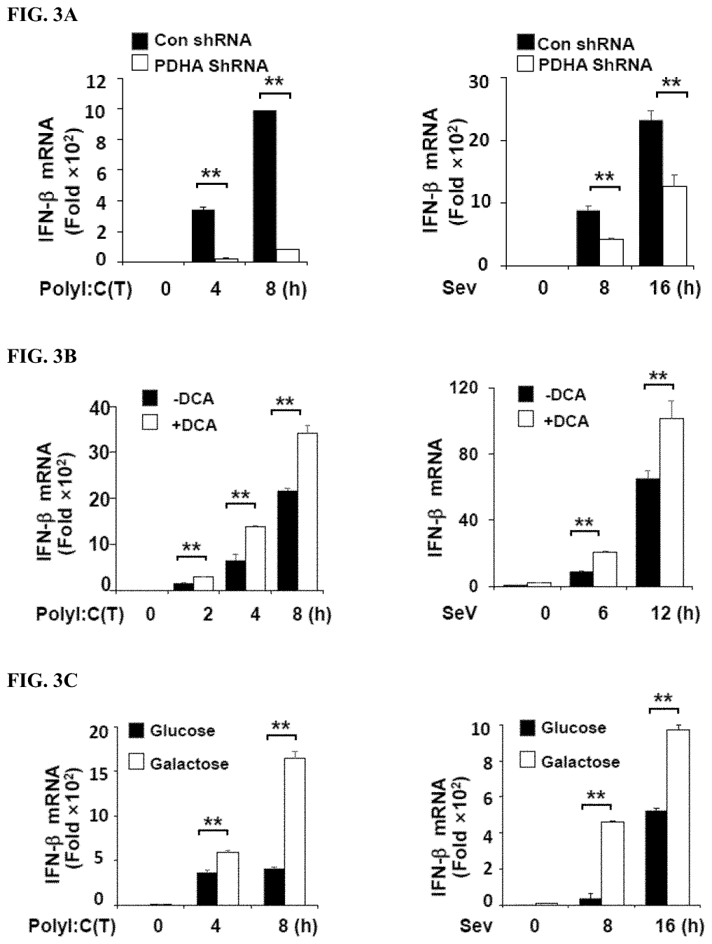

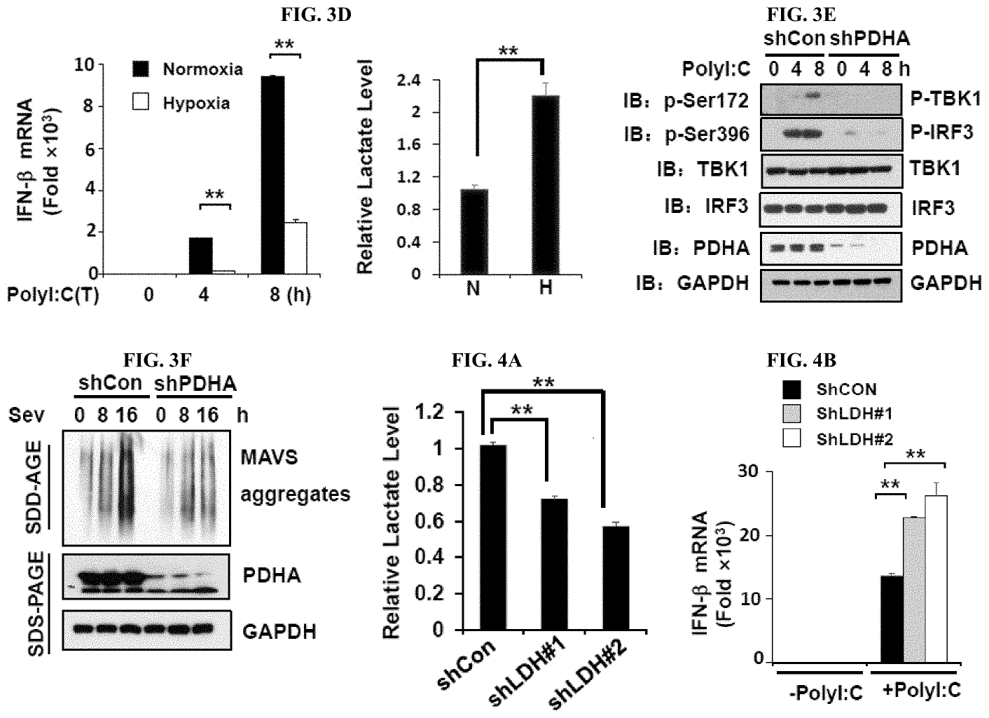

As the two major glucose catabolism pathways, oxidative phosphorylation (OXPHOS) and anaerobic glycolysis are tightly controlled in cells by a multistep mechanism to execute diverse biological processes (Lunt and Vander Heiden, 2011). It was shown that downregulation of glucose metabolism is required for full activation of RLR signaling. To dissect the key step of glucose metabolism involved in regulating type-I IFN production, various strategies were adapted to challenge glucose metabolism either by triggering the switch from OXPHOS to anaerobic glycolysis or vice versa. Metabolic fate of pyruvate is mainly controlled by LDHA and PDHc, which convert pyruvate to lactate or acetyl-CoA for TCA cycle, respectively (Ganeshan and Chawla, 2014). As the crucial PDHc subunit, PDHA knockdown impaired mitochondria OXPHOS, thus promoting anaerobic glycolysis for lactate production. Interestingly, robust decrease of IFN-β production in PDHA knockdown cells compared with control cells was found upon RLR activation (FIG. 3A). Similarly, the treatment of cells with UK5099, a potent inhibitor of the mitochondria pyruvate transporter blocking pyruvate mitochondria entry for OXPHOS (Davies et al., 2017; Liu et al., 2018), elevated lactate level and led to decreased MAVS signaling and IFN-β induction (data not shown). By contrast, dichloroacetate (DCA), which shifts glucose metabolism from anaerobic glycolysis to OXPHOS by inhibiting pyruvate dehydrogenase kinase and thus activating PDHc (Baker et al., 2000; Michelakis et al., 2008), promoted IFN-β induction upon RLR activation, accompanied by the reduction of lactate level (FIG. 3B). Cells grown in galactose conditions are forced to respire by using OXPHOS instead of anaerobic glycolysis (Chang et al., 2013; Rossignol et al., 2004; Weinberg et al., 2010). While galactose expectedly impaired anaerobic glycolysis-mediated lactate production, it markedly enhanced IFN-β and IL-6 production upon poly(I:C) or Sev challenge (FIG. 3C). VSV-GFP-infected iBMM cells pre-cultured in the same conditions as in FIG. 3C and then infected with VSV-GFP (MOI=0.1) for 0 hr, 6 hr, and 12 hr were assessed. Such cells showed a marked reduction in viral replication, as determined by GFP fluorescence, under galactose treatment (no GFP expression under Galactose treatment vs increasing GFP expression at 6 hr and 12 hr under Glucose treatment; data not shown). These data suggest that anaerobic glycolysis serves as a negative signal to repress RLR-mediated MAVS signaling and cytokine production.

To further investigate the biological role of anaerobic glycolysis in RLR signaling activation, cells were grown under hypoxia-inducing conditions, which physiologically promotes anaerobic glycolysis. While hypoxia challenge expectedly induced the expression of HIF1α targeted genes, such as VEGF and GLUT4, and promoted lactate generation, it markedly reduced IFN-β production upon RLR activation (FIG. 3D). Upon recognition of cytosolic RNA, RLR undergoes an ATP-dependent conformational change that facilitates MAVS oligomerization, which triggers TBK1 activation and subsequent IRF3 phosphorylation for IFN-β production. To understand how anaerobic glycolysis regulates type-I IFN production during RLR activation, the phosphorylation of TBK1 and IRF3 was examined. Consistent with decreased IFN-β expression, the levels of TBK1 and IRF3 phosphorylation robustly declined in PDHA knockdown cells in response to poly(I:C) transfection (FIG. 3E). Moreover, it was demonstrated that PDHA knockdown attenuated MAVS aggregation upon Sev infection (FIG. 3F). Taken together, these data suggest that anaerobic glycolysis suppresses RLR induced downstream activation by affecting MAVS or upstream function.

### Example 5. LDHA Associated Lactate Negatively Regulates RLR Signaling

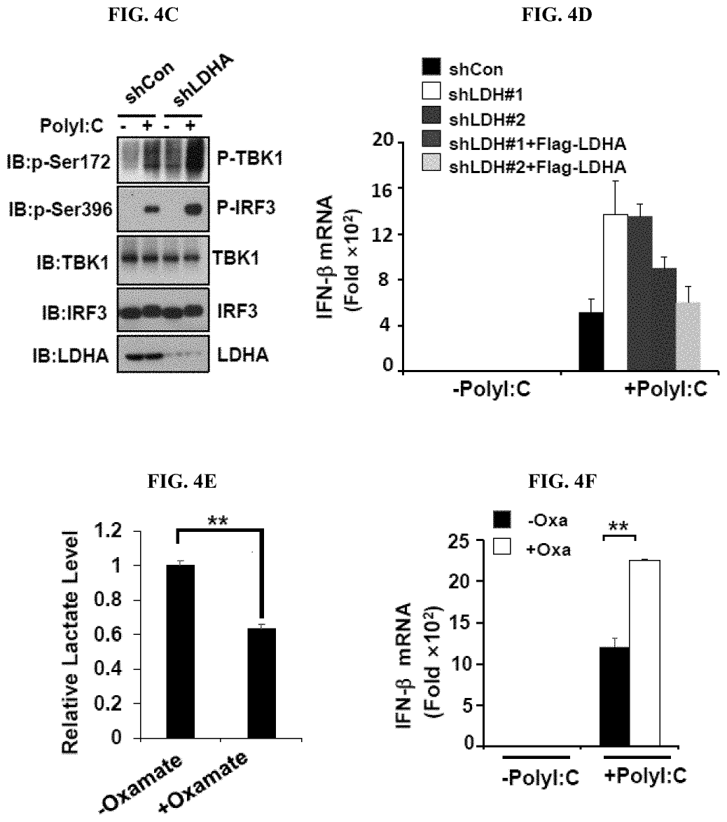

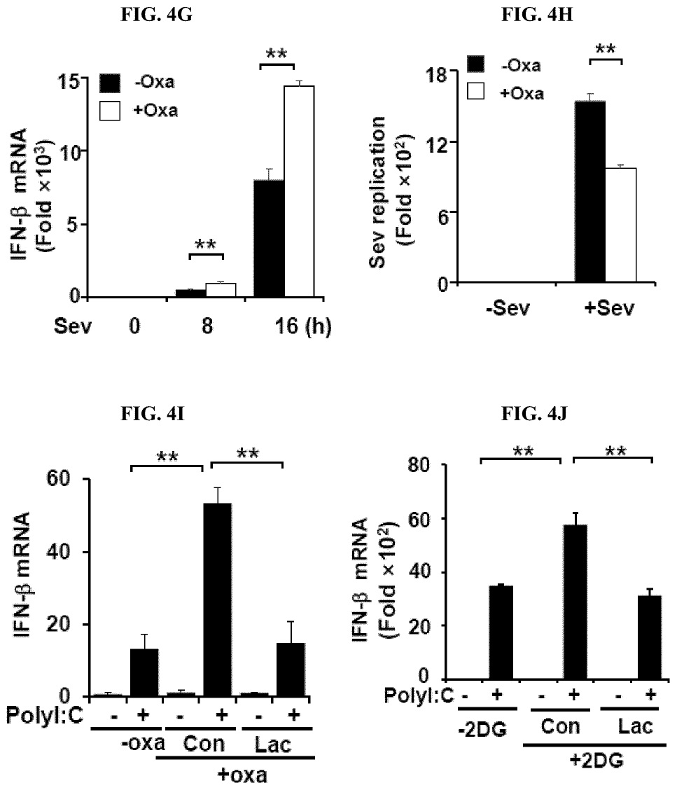

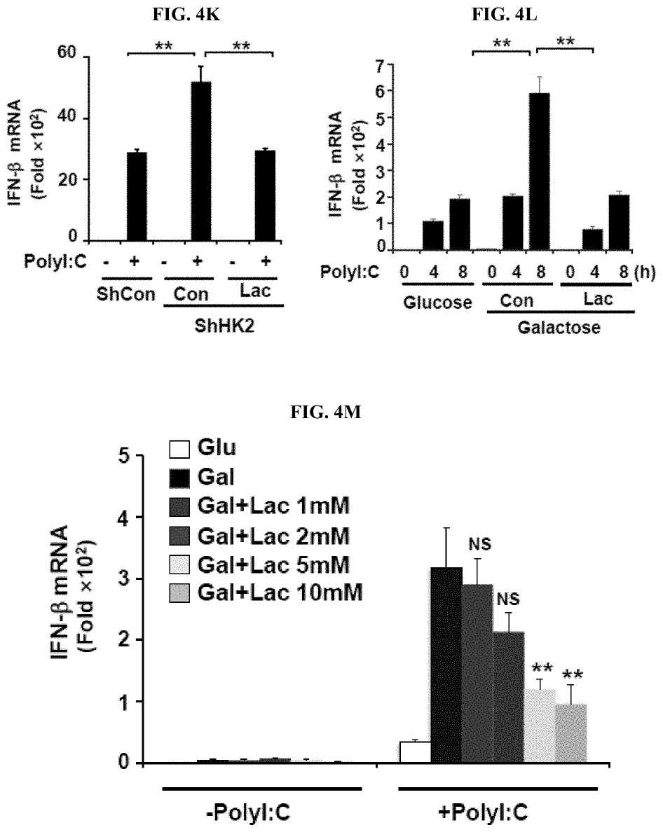

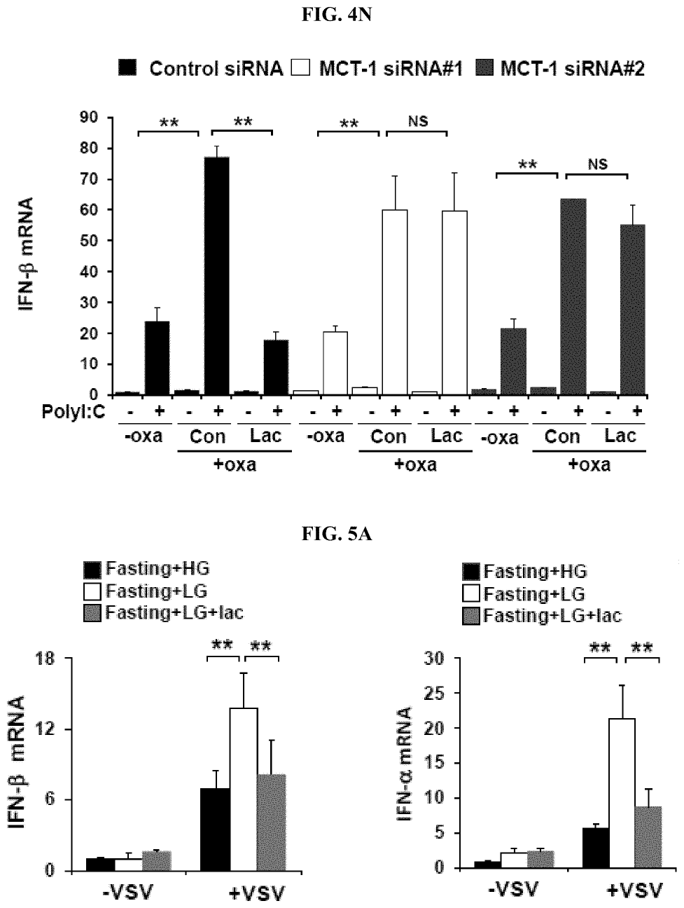

Since LDHA catalyzing lactate generation from pyruvate is a key event for anaerobic glycolysis, genetic and pharmacologic approaches were employed to determine whether LDHA-associated lactate plays a role in negatively regulating type-I IFN. Indeed, knockdown of LDHA enhanced phosphorylation of TBK1 and IRF3 as well as IFN-β production upon RLR activation (FIG. 4A-4C). Importantly, LDHA restoration in these knockdown cells reversed this effect (FIG. 4D). Similar to LDHA knockdown, treatment of sodium oxamate, a specific LDHA inhibitor (Crane et al., 2014; Zhao et al., 2014; Zhao et al., 2016), reduced lactate level and enhanced IFN-β production resulting in suppressing virus replication (FIG. 4E-4H). Sodium oxamate also promoted IRF3 and TBK1 phosphorylation and MAVS aggregation upon RLR activation (data not shown). Of note, the effect of LDHA inhibition on RLR signaling was not observed in cells treated by STING agonist. Collectively, these results indicate that LDHA plays a crucial role in specifically suppressing RLR-MAVS signaling and subsequent type-I IFN production.

Next, experiments were conducted to determine whether LDHA-associated lactate is a direct metabolite meditating the inhibition of RLR signaling caused by anaerobic glycolysis. Of note, addition of lactate into cells pretreated with sodium oxamate reversed the effect of LDHA inhibitor on IFN-β promotion (FIG. 4I). Lactate add-back also compromised the effects of 2-DG on IFN-β induction and its upstream signaling (FIG. 4J). Similar results were also observed in HK2 knockdown cells (FIG. 4K). Moreover, it was shown that add-back of lactate rescued the effects of galactose on IFN-β induction and virus replication (FIG. 4L). To determine how much lactate is needed to inhibit IFN production, various concentrations of exogenous lactate (0-10 mM) were added back to cells treated with galactose, and 5 mM lactate was required for inhibiting IFN-β production (FIG. 4M). To rule out the possibility that lactate affects IFN-β production by changing the acidity of the medium, acidic pH of medium was adjusted to equal level as cells without addition of lactate by using NaOH before Poly(I:C) transfection. Lactate could still inhibit IFN-β production to the similar extent as the condition where PH was not adjusted (data not shown). As cells utilize monocarboxylate transporter 1 (MCT1) for lactate uptake, MCT1 was knocked down in cells to determine whether lactate uptake is crucial for its effect. LDHA inhibitor-Oxamate or 2-DG treatment promoted IFN-β expression, but such effect was markedly impaired upon MCT1 knockdown (FIG. 4N), indicating that lactate transporting into cells is essential for inhibiting MAVS signaling. Collectively, it can be concluded that lactate serves as a key metabolite to mediate the inhibitory effect of anaerobic glycolysis on RLR signaling.

### Example 6. LDHA-Associated Lactate Inhibits RLR Signaling In Vivo

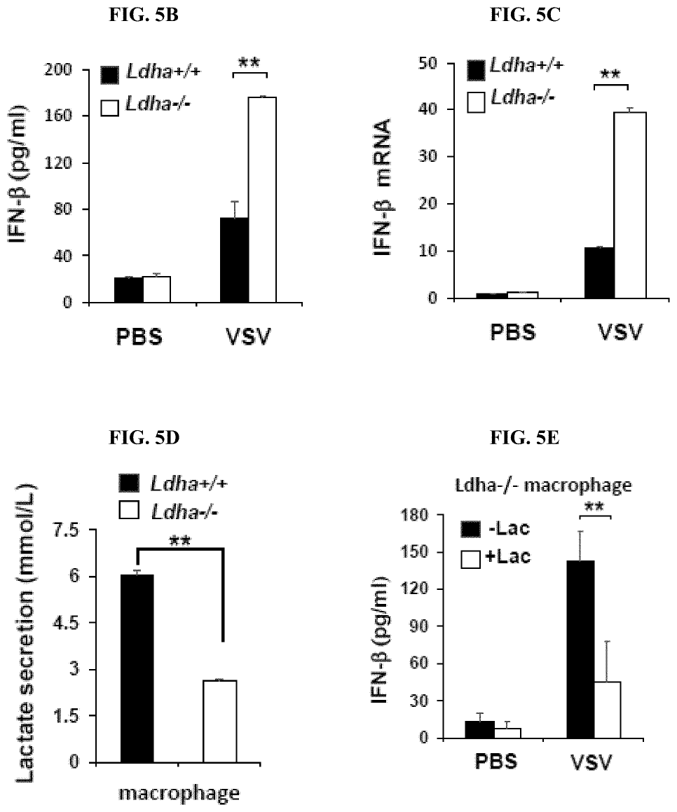

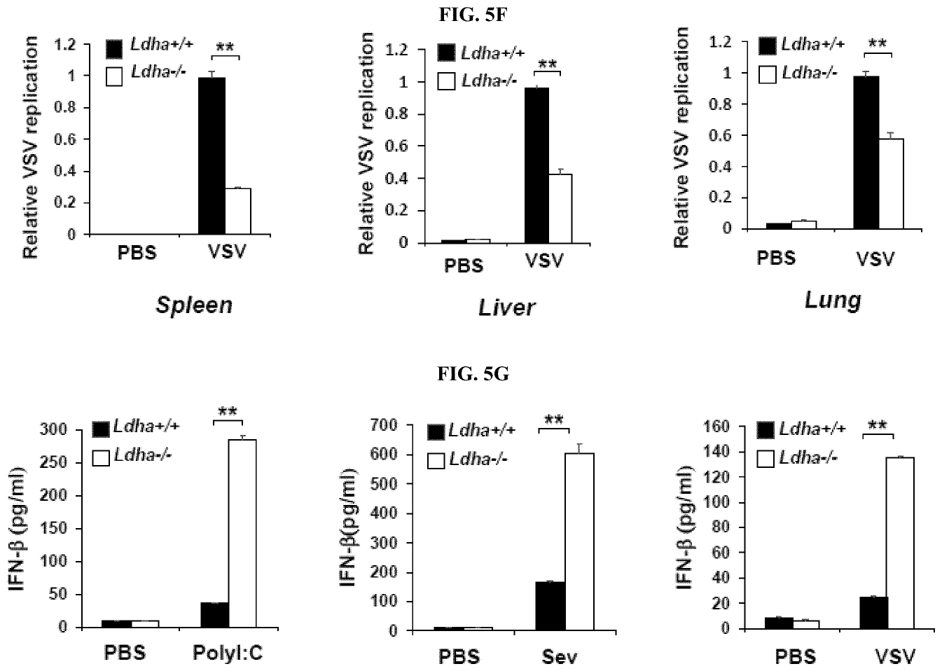

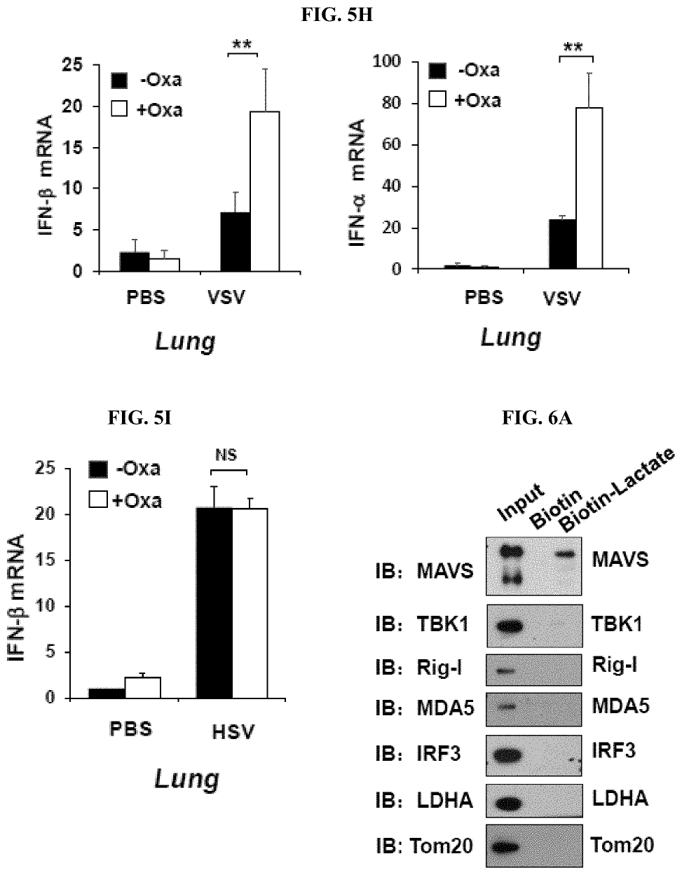

To further confirm glycolysis-derived lactate serves as a natural suppressor of RLR induced type-I IFN production in vivo, mice were fasted overnight to reduce the basal glucose and lactate level and then treated with high glucose or low glucose in the presence or absence of lactate addition. While the reduction of glucose level in mice enhanced type-I IFN and IL-6 induction triggered by VSV infection, lactate add-back reversed this effect (FIG. 5A), indicating that lactate plays a critical role in suppressing RLR signaling in vivo. Moreover, tamoxifen inducible LDHA knockout mice and primary macrophage cells, in which LDHA exon2 is flanked by loxP sites (Xie et al., 2014) and lactate generation is genetically inhibited, were used to monitor RLR-mediated cytokine production. LDHA protein expression in diverse organs of Ldha^{-/-} mice was abrogated upon tamoxifen treatment (data not shown). When challenged with VSV, Ldha^{-/-} mice showed much higher cytokine induction in serum or tissue compared to that in Ldha^{+/+} mice (FIG. 5B-5C). Consistent with elevated IFN-β level, virus replication as determined by VSV-specific mRNA in different tissues was much lower in Ldha^{-/-} mice than Ldha^{+/+} mice (FIG. 5F). Pathological staining showed reduced infiltration of inflammatory cells in the lung of Ldha^{-/-} mice compared with Ldha^{+/+} mice upon VSV infection (data not shown). In addition, peritoneal macrophages isolated from Ldha^{-/-} mice exhibited reduced lactate secretion, but elevated IFN-β induction compared with those from Ldha^{+/+} mice upon poly (I:C) transfection, Sev or VSV infection, whereas no difference was observed between the groups when treated by HTDNA or HSV (FIG. 5D and FIG. 5G). Moreover, lactate addition in Ldha^{-/-} macrophage consistently reduced IFN-β production (FIG. 5E). These data define the critical role of LDHA-associated lactate in suppressing RLR-mediated type-I IFN production for the host defense against viral infection in vivo.

### Example 7. Pharmacologically Targeting LDHA-Associated Lactate Enhances Type-I IFN Production and Viral Clearance In Vivo

Having shown that LDHA-mediated lactate serves as a nature inhibitor of RLR-mediated type-I IFN production and viral clearance in vivo, it was next determined whether pharmacologically targeting LDHA by the LDHA inhibitor, sodium oxamate, in mice is an effective strategy to bolster IFN production for the host to defend against viral infection (data not shown). Remarkably, administration of sodium oxamate to mice reduced lactate production and promoted the induction of type-I IFN, IL-6 and virus clearance in various tissue samples after VSV infection (FIG. 5H). The effect of lactate on IFN production in vivo is specific for RLR, because no difference in IFN production was observed in mice injected with HSV that activates STING signaling (FIG. 5I). Thus, these results provide the proof of principle evidence that pharmacologically targeting LDHA by sodium oxamate is an effective strategy to enhance RLR triggered type-I IFN production for viral clearance.

### Example 8. LDHA-Associated Lactate Negatively Regulates RLR Activation by Targeting MAVS

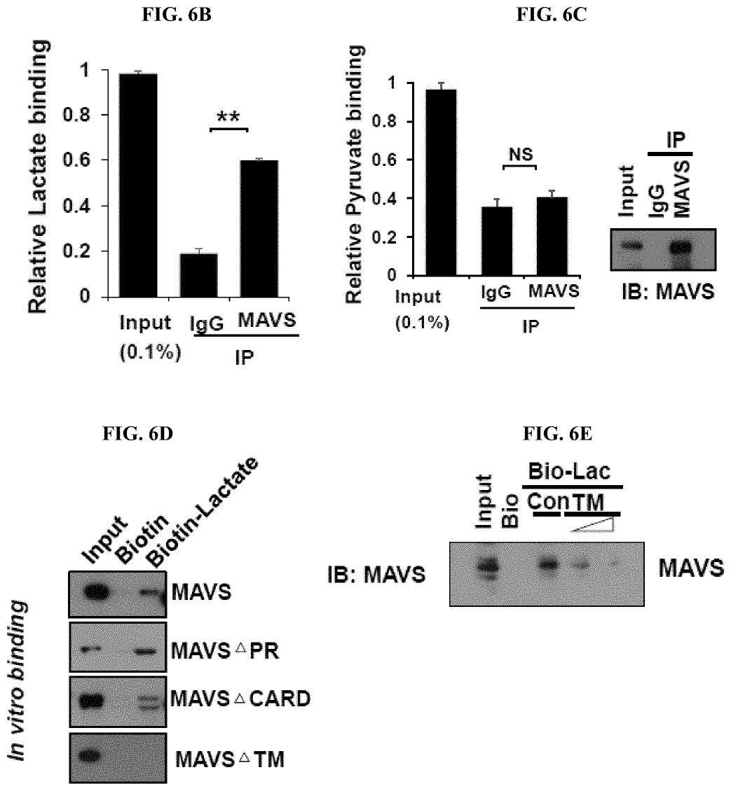

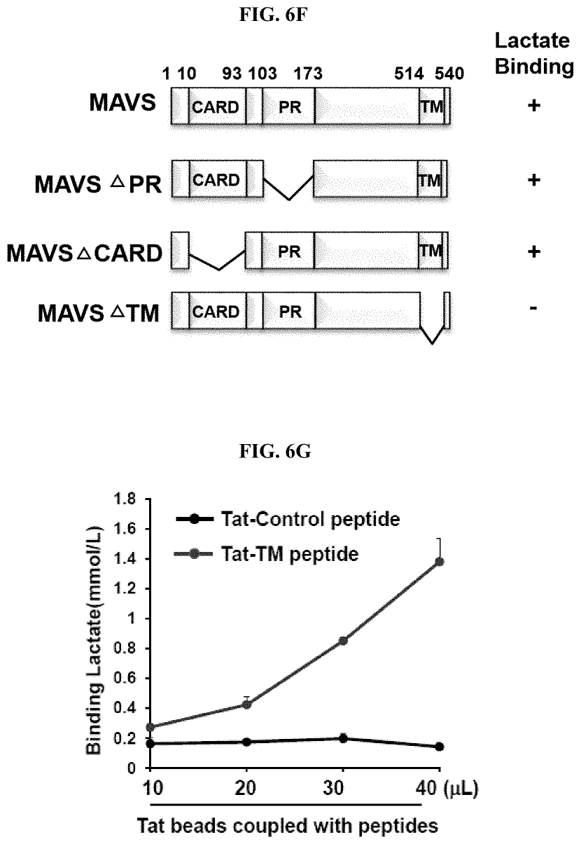

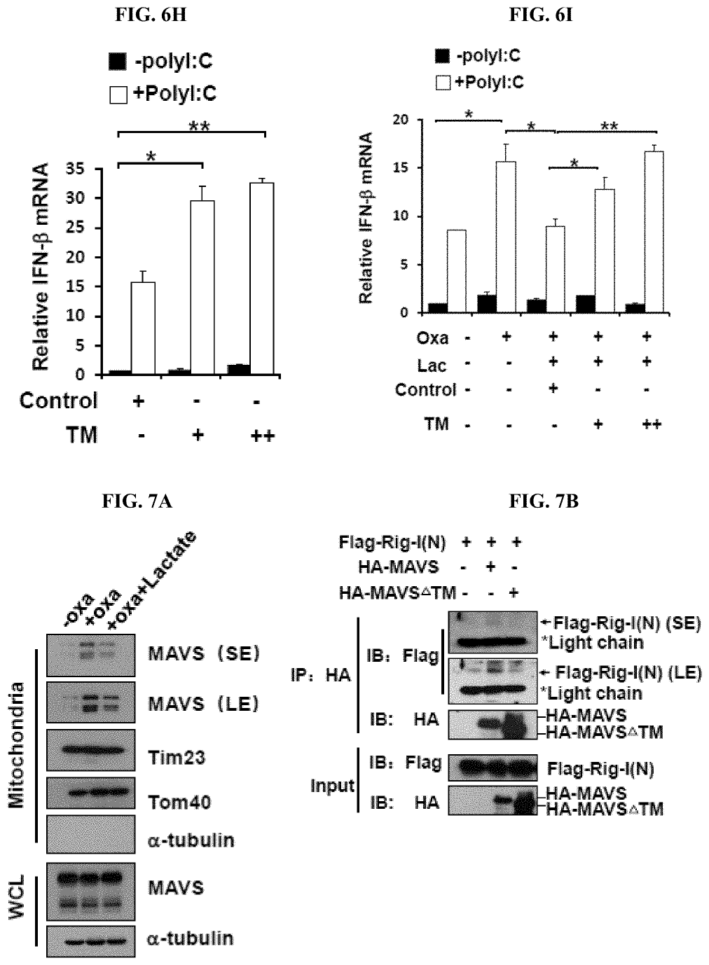

Additional experiments were conducted to identify the direct targets of lactate responsible for its action on RLR-signaling. To this end, biotin-labeled lactate was synthesized and biotin pulldown assays were performed by mixing biotin-labeled lactate with the whole cell extracts. Assay measurements focused on the key proteins that are involved in cytosolic RLR signaling activation including RIG-I, MDAS, TBK1, MAVS and IRF3. It was found that lactate specifically interacted with MAVS, but not with other components of cytosolic RLR signaling or other mitochondria proteins such as Tom20 (FIG. 6A), indicative of the specificity of this interaction. Mass spectrometry analysis also identified a cluster of MAVS peptides in the lactate pulldown complex (data not shown). Of note, MAVS interacted with lactate, but not pyruvate, in cells under physiological conditions (FIGS. 6B and 6C). By using in vitro binding assays, MAVS was found to bind to lactate in a dose-dependent manner. Pre-incubation with excessive lactate significantly attenuated the binding of MAVS to Biotin-lactate (data not shown). Collectively, these data suggest that lactate directly binds to MAVS.

To explore the detailed interaction between lactate and MAVS, domain mapping assays were carried out and the transmembrane (TM) domain of MAVS that targets MAVS to the mitochondrial membrane was found to be necessary for lactate-MAVS binding (FIG. 6D and FIG. 6F). Next, a peptide containing MAVS TM domain (514-535aa) with a trans-activator of transcription (TAT) tag placed in its N terminal region was synthesized (SEQ ID NO:24) along with a corresponding control construct (SEQ ID NO:43). In an in vitro binding assay, it was found that TM peptide markedly interrupted the binding of lactate to MAVS in dose-dependent manner (FIG. 6E), indicating this peptide could compete with MAVS from binding to lactate. Experiments were conducted to determine whether lactate could directly bind to TM domain of MAVS. In support of this notion, mass spectrometry analysis of in vitro biotin-lactate pulldown products revealed that the TM peptide of MAVS, but not control peptide, could bind to biotin-lactate (data not shown). Furthermore, a Tat antibody was used to perform immunoprecipitation assays and it was discovered that lactate could be specifically pulled down by Tat-TM peptide in a dose-dependent manner (FIG. 6G). Thus, these data demonstrate that lactate binds to TM domain of MAVS.

The Tat peptide derived from the trans-activator of transcription of human immunodeficiency virus is known to possess cell penetration properties (Milletti, 2012). This property has been used to deliver large molecules or even small particles into the cells for testing their biological functions by overcoming the lipophilic barrier of the cell membranes. Cellular localization of the control and TM peptides was assessed using the immunofluorescence assay in which HeLa cells were pretreated with the peptides and then stained with MAVS or Tat antibody and then imaged by confocal microscopy. Both the control and TM peptide were found to enter into cells (similar cytoplasmic staining; data not shown). The effect of TM peptide on RLR induced type-I IFN production was then determined. Of note, adding TM peptide to the cells promoted RLR activation in a dose dependent fashion (FIG. 6H). Moreover, TM peptide also compromised the inhibitory effect of lactate on IFN-β and IL-6 production upon Poly (I:C) treatment (FIG. 6I). Collectively, these results suggest that lactate inhibits RLR signaling through its binding to the TM domain of MAVS.

### Example 9. Lactate Inhibits MAVS Mitochondria Localization, RIG-I/MAVS Association and MAVS Aggregation

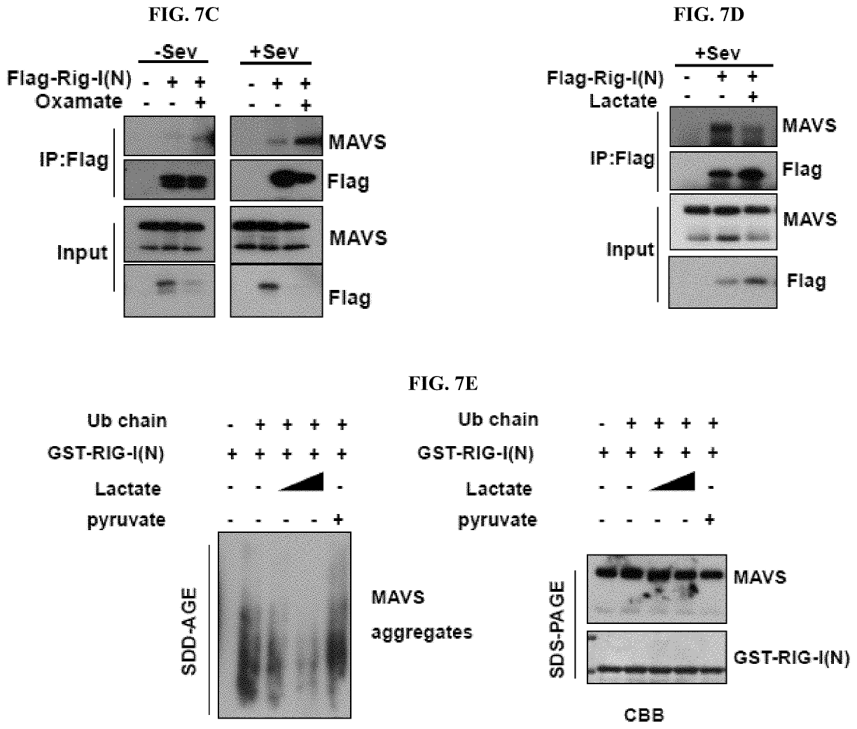

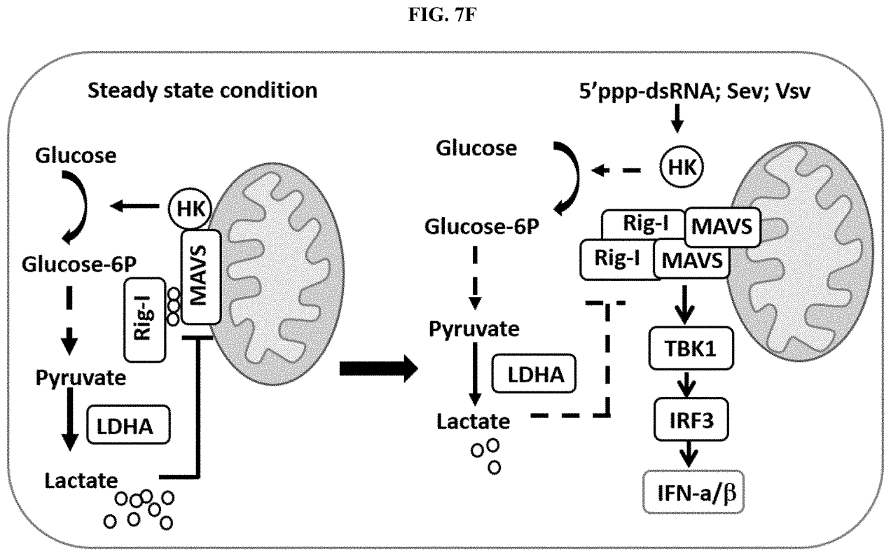

In order to unravel the underlying mechanism by which lactate inhibits RIG-I-MAVS induced RLR activation, immunoprecipitation and immunofluorescence localization experiments were conducted to explore whether lactate impacts the mitochondria localization of MAVS by binding to its TM domain (Seth et al., 2005). These experiments showed that inhibition of lactate generation by oxamate potentiated MAVS mitochondria localization, but lactate add-back compromised this effect. This is shown in FIG. 7A by the increased in MAVS band intensity with the addition of oxamate, which was reduced when both oxamate and lactate were present. Similarly, addition of oxamate resulted in strong mitochondrial staining of MAVS, which was reduced to background levels when both oxamate and lactate were added (IF data not shown). Mitochondria localization of MAVS provides the structural basis for RIG-I-MAVS recognition and subsequent MAVS activation. It was then determined whether activated RIG-I could still bind to MAVS devoid of TM domain of MAVS. As expected, MAVS mutant defective of TM domain which could not localize in mitochondria failed to interact with activated RIG-I (FIG. 7B). Notably, LDHA inhibitor treatment enhanced the interaction between activated RIG-I and MAVS in both basal and Sendai virus-infected conditions, whereas lactate treatment markedly reduced their binding (FIGS. 7C and 7D). Thus, lactate disrupts MAVS mitochondria localization and the interaction between RIG-I-MAVS interaction.

Recognition of MAVS by activated RIG-I is understood to be essential for MAVS aggregation and its downstream activation. Tests were conducted to determine whether lactate could directly affect MAVS aggregation induced by activated RIG-I through performing in vitro MAVS aggregation assay. To this end, lactate was incubated with activated RIG-I protein (RIG-I(N)) along with mitochondria and ubiquitin chains as previously described (Hou et al., 2011). In the presence of mitochondria and unanchored K63-linked ubiquitin chains (K63-Ub4), purified GST-RIG-I(N) was found to trigger robust MAVS aggregation in vitro. Remarkably, lactate, but not pyruvate, impaired MAVS aggregation in a dose dependent manner (FIG. 7E), indicative of direct effect of lactate on RIG-I-mediated MAVS aggregation. Collectively, these data suggest that lactate targets the TM domain of MAVS to obstruct its mitochondria localization and RIG-I-MAVS complex formation, thereby impairing MAVS aggregation and its downstream signaling activation.

Substantial evidence accumulated in recent years has highlighted the role of lactate not only in regulating tumor microenvironment and immune cell functions (Brand et al., 2016; Colegio et al., 2014), but also serving as a fuel for TCA cycle in cancer cells (Faubert et al., 2017). However, no direct targeting protein of lactate has been identified so far to help explain its mode of action. The studies described herein identify MAVS as a lactate sensor whose inactivation by direct lactate binding serves as a natural barrier to limit RLR signaling activation for type-I IFN production. It is clear that the binding of MAVS to lactate interrupts MAVS mitochondria localization, RIG-I and MAVS interaction and subsequent MAVS aggregation, thereby attenuating RLR signaling and downstream type-I IFN production (FIG. 7F).

Although metabolism has been linked to diverse biological processes, the connection between glucose metabolism and RLR-mediated innate immune activation remains puzzling. Through systematic approaches in conjunction with various biochemical assays, it was discovered that anaerobic glycolysis acts as a natural barrier to impede RLR signaling activation. Importantly, glycolysis-derived lactate represents the first metabolite that directly binds to MAVS and suppresses its functions to orchestrate type-I IFN production. Based on these findings, it is now believed that lactate accumulation by elevated LDH and/or glycolysis may be a potential mechanism for viruses to evade host defense by inhibiting RLR-induced type-I IFN production. Notably, clinical studies reveal that elevated LDH or lactate levels are detected in patients under certain viral infection, especially in those with poor prognosis (Chen et al., 2013b; Hunt et al., 2015). Hence, pharmacologically targeting LDHA-dependent lactate production is a promising strategy to strengthen host innate immune response through heightening type-I IFN production for viral clearance and cancer immune-surveillance.

TM domain of MAVS is not only crucial for the mitochondria localization of MAVS, but also required for RIG-I recognition. Interestingly, the finding that TM peptide competes with MAVS from binding to lactate and lactate directly binds to TM domain of MAVS may provide the molecular basis of how lactate disrupts MAVS mitochondria localization and MAVS-RIG-I interaction, thereby impairing MAVS aggregation and RLR-mediated IFN production for viral clearance. Of note, applying TM peptide from MAVS to the cells not only enhances IFN production under normal conditions, but also relieves the inhibitory effect of lactate on RLR-mediated IFN production. Thus, these findings suggest that targeting lactate-MAVS interaction may represent another promising strategy for clinical drug development based on the knowledge learned from TM peptide to boost the body's immunity for viral and cancer clearance. This strategy may offer a superior way than directly targeting LDHA, as it maintains the level of lactate for normal cellular functions.

Type-I IFNs are critical cell-intrinsic antimicrobial factors that limit the spread of infectious agents, such as viral pathogens. (MacMicking, 2012; Schoggins et al., 2011; Stark and Darnell, 2012). Beyond their important role in host defense against viruses, type-I IFNs also play a critical role in regulating the functions of diverse innate and adaptive immune cells including dendritic cell, regulatory T (TReg) cells and cytotoxic T lymphocytes (CTLs), thereby contributing to immune-surveillance of cancer (Zitvogel et al., 2015). While energy metabolism has emerged to regulate diverse biological processes, its role in RLR signaling activation and type-I IFN production has not been defined. The studies described herein reveal that glycolysis serves as physiological barriers to limit type-I IFN production by promoting lactate generation, both in vitro and in vivo. Hence, in order to achieve optimal type-I IFN production, it is imperative for the host to develop a strategy to inactivate glycolysis and thereby reducing lactate production during RLR challenge. In this respect, the RLR challenge was demonstrated to switch the recognition of MAVS from HK2 to RIG-I, resulting in HK2 mitochondria dissociation and HK inactivation. This study therefore uncovers that MAVS is a previously unrecognized player that maintains HK2 activity and glycolysis. Thus, MAVS possess dual roles in regulating glycolysis and innate immune response, and viral infection hijacks MAVS's function from glycolysis to innate immune regulation.

Since cancer cells often overexpressed HK2 (Patra et al., 2013), exposed to hypoxia microenvironment and favored glycolysis for energy metabolism known as Warburg effect (Nakazawa et al., 2016; Pavlova and Thompson, 2016), it is likely that limiting type-I IFN production by these conditions through generating more lactate may serve as another important mechanism for cancer cells to evade immune-surveillance. In summary, the studies described herein provide not only the crucial molecular insight into how energy metabolism and type-I IFN cross-talk to regulate diverse biological processes, but also offer an important paradigm and strategy for the management of various human diseases, such as viral infection and cancer.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A peptide construct comprising a mitochondrial antiviral-signaling protein (MAVS) peptide and a cell penetration peptide.

2. The peptide construct of claim 1, wherein the MAVS peptide comprises a MAVS transmembrane domain.

3. The peptide construct of claim 2, wherein the MAVS transmembrane domain comprises an amino acid sequence having at least 70% identity to SEQ ID NO:2.

4. The peptide construct of claim 1, wherein the cell penetration peptide is an HIV-1 Tat peptide.

5. The peptide construct of claim 4, wherein the HIV-1 Tat peptide comprises an amino acid sequence having at least 70% identity to SEQ ID NO:6.

6. The peptide construct of claim 1, wherein the cell penetration peptide comprises the amino acid sequence of SEQ ID NO:6 and the MAVS peptide comprises an amino acid sequence having at least 70% identity to SEQ ID NO:2.

7. The peptide construct of claim 1, wherein the C-terminus of the cell penetration peptide is linked to the N-terminus of the MAVS peptide.

8. The peptide construct of claim 1, comprising the amino acid sequence of SEQ ID NO:24.

9. A nucleic acid encoding a peptide construct according to claim 1.

10. A vector comprising the nucleic acid of claim 9.

11. A host cell comprising the nucleic acid of claim 9.

12. A host cell comprising the vector of claim 10.

13. A pharmaceutical composition comprising a peptide construct according to claim 1 and a pharmaceutically acceptable excipient.

14. A method of stimulating interferon production in a cell, the method comprising contacting the cell with an effective amount of a peptide construct according to claim 1, a hexokinase inhibitor, a lactate dehydrogenase inhibitor, or a combination thereof.

15. A method of treating a viral infection, the method comprising administering to a subject in need thereof a therapeutically effective amount of a peptide construct according claim 1.

16. The method of claim 15, wherein the viral infection is caused by an RNA virus.

17. The method of claim 15, wherein the viral infection is a parainfluenza virus infection.

18. The method of claim 15, further comprising administering to the subject a lactate dehydrogenase (LDH) inhibitor, a hexokinase (HK) inhibitor, an antiviral agent, or a combination thereof.

19. The method of claim 18, wherein the LDH inhibitor is sodium oxamate.

20. The method of claim 18, wherein the HK inhibitor is 2-deoxyglucose.

21. A method of treating cancer, the method comprising administering to a subject in need thereof a therapeutically effective amount of a peptide construct according to claim 1.

22. The method of claim 21, further comprising administering an anti-cancer agent to the subject.

23. The method of claim 21, further comprising administering radiation therapy to the subject.

24. The method of claim 21, further comprising administering to the subject a lactate dehydrogenase (LDH) inhibitor, a hexokinase (HK) inhibitor, or a combination thereof.

25. The method of claim 24, wherein the LDH inhibitor is sodium oxamate.

26. The method of claim 24, wherein the HK inhibitor is 2-deoxyglucose.


SEQUENCE LISTING (43 sequences)

SEQ ID NO: 1 - protein, 540 aa, Homo sapiens [sequence omitted]

SEQ ID NO: 2 - protein, 22 aa, Homo sapiens
GALWLQVAVTGVLVVTLLVVLY

SEQ ID NO: 3 - protein, 22 aa, Unknown
  Description of Unknown N. leucogenys, H. albibarbis, or S. syndactylus MAVS sequence
GALWLQVAVAGVLAVTLLVVLY

SEQ ID NO: 4 - protein, 22 aa, Unknown
  Description of Unknown M. mulatta, P. anubis, or T. gelada MAVS sequence
GALWLQAAVAGVLVVTLLVAMY

SEQ ID NO: 5 - protein, 22 aa, Unknown
  Description of Unknown C. capucinus imitator or C. jacchus MAVS sequence
GSPWLQAAVAGVLAAILLAVLY

SEQ ID NO: 6 - protein, 12 aa, Human immunodeficiency virus 1
YGRKKRRQRRRA

SEQ ID NO: 7 - protein, 13 aa, Human immunodeficiency virus 1
GRKKRRQRRRPPQ

SEQ ID NO: 8 - protein, 9 aa, Human immunodeficiency virus 1
RKKRRQRRR

SEQ ID NO: 9 - protein, 16 aa, Drosophila melanogaster
RQIKIWFQNRRMKWKK

SEQ ID NO: 10 - protein, 19 aa, Artificial Sequence
  Description of Artificial Sequence Synthetic peptide
VKRGLKLRHVRPRVTRMDV

SEQ ID NO: 11 - protein, 27 aa, Artificial Sequence
  Description of Artificial Sequence Synthetic peptide
GALFLGFLGAAGSTMGAWSQPKKKRKV

SEQ ID NO: 12 - protein, 21 aa, Artificial Sequence
  Description of Artificial Sequence Synthetic peptide
KETWWETWWTEWSQPKKKRKV

SEQ ID NO: 13 - protein, 18 aa, Mus sp.
LLIILRRRIRKQAHAHSK

SEQ ID NO: 14 - protein, 22 aa, Artificial Sequence
  Description of Artificial Sequence Synthetic peptide
MVRRFLVTLRIRRACGPPRVRV

SEQ ID NO: 15 - protein, 28 aa, Bos sp.
MVKSKIGSWILVLFVAMWSDVGLCKKRP

SEQ ID NO: 16 - protein, 18 aa, Artificial Sequence
  Description of Artificial Sequence Synthetic peptide
KLALKLALKALKAALKLA

SEQ ID NO: 17 - protein, 27 aa, Artificial Sequence
  Description of Artificial Sequence Synthetic peptide
GWTLNSAGYLLGKINLKALAALAKKIL

SEQ ID NO: 18 - protein, 28 aa, Unknown
  Description of Unknown Azurin sequence
LSTAADMQGVVTDGMASGLDKDYLKPDD

SEQ ID NO: 19 - protein, 26 aa, Artificial Sequence
  Description of Artificial Sequence Synthetic peptide
DPKGDPKGVTVTVTVTVTGKGDPKPD

SEQ ID NO: 20 - protein, 24 aa, Artificial Sequence
  Description of Artificial Sequence Synthetic peptide
RRIRPRPPRLPRPRPRPLPFPRPG

SEQ ID NO: 21 - protein, 17 aa, Artificial Sequence
  Description of Artificial Sequence Synthetic peptide
CSIPPEVKFNKPFVYLI

SEQ ID NO: 22 - protein, 6 aa, Artificial Sequence
  Description of Artificial Sequence Synthetic peptide
PFVYLI

SEQ ID NO: 23 - protein, 15 aa, Artificial Sequence
  Description of Artificial Sequence Synthetic peptide
SDLWEMMMVSLACQY

SEQ ID NO: 24 - protein, 34 aa, Artificial Sequence
  Description of Artificial Sequence Synthetic polypeptide
YGRKKRRQRRRAGALWLQVAVTGVLVVTLLVVLY

SEQ ID NO: 25 - DNA, 20 bp, Artificial Sequence
  Description of Artificial Sequence Synthetic primer
tccaaattgctctcctgttg

SEQ ID NO: 26 - DNA, 20 bp, Artificial Sequence
  Description of Artificial Sequence Synthetic primer
gcagtattcaagcctcccat

SEQ ID NO: 27 - DNA, 22 bp, Artificial Sequence
  Description of Artificial Sequence Synthetic primer
tccgagcagagatcttcaggaa

SEQ ID NO: 28 - DNA, 22 bp, Artificial Sequence
  Description of Artificial Sequence Synthetic primer
tgcaaccaccactcattctgag

SEQ ID NO: 29 - DNA, 24 bp, Artificial Sequence
  Description of Artificial Sequence Synthetic primer
tgcagaaaaaggcaaagaatctag

SEQ ID NO: 30 - DNA, 19 bp, Artificial Sequence
  Description of Artificial Sequence Synthetic primer
cgtcagcaggctggcattt

SEQ ID NO: 31 - DNA, 22 bp, Artificial Sequence
  Description of Artificial Sequence Synthetic primer
agttgccttcttgggactgatg

SEQ ID NO: 32 - DNA, 25 bp, Artificial Sequence
  Description of Artificial Sequence Synthetic primer
gggagtggtatcctctgtgaagtct

SEQ ID NO: 33 - DNA, 24 bp, Artificial Sequence
  Description of Artificial Sequence Synthetic primer
cctgaacatcttcacatcaaagga

SEQ ID NO: 34 - DNA, 20 bp, Artificial Sequence
  Description of Artificial Sequence Synthetic primer
agctgctggtggaggtcatt

SEQ ID NO: 35 - DNA, 20 bp, Artificial Sequence
  Description of Artificial Sequence Synthetic primer
gagtcaacggatttggtcgt

SEQ ID NO: 36 - DNA, 20 bp, Artificial Sequence
  Description of Artificial Sequence Synthetic primer
ttgattttggagggatctcg

SEQ ID NO: 37 - DNA, 21 bp, Artificial Sequence
  Description of Artificial Sequence Synthetic primer
cagtcccagcgtcgtgattag

SEQ ID NO: 38 - DNA, 23 bp, Artificial Sequence
  Description of Artificial Sequence Synthetic primer
aaacactttttccaaatcctcgg

SEQ ID NO: 39 - DNA, 20 bp, Artificial Sequence
  Description of Artificial Sequence Synthetic primer
gctgccgacaaggtgagagc

SEQ ID NO: 40 - DNA, 20 bp, Artificial Sequence
  Description of Artificial Sequence Synthetic primer
gcccgccatgcctctctcta

SEQ ID NO: 41 - DNA, 19 bp, Artificial Sequence
  Description of Artificial Sequence Synthetic primer
acggcgtacttccagatgg

SEQ ID NO: 42 - DNA, 19 bp, Artificial Sequence
  Description of Artificial Sequence Synthetic primer
ctcggttcaagatccaggt

SEQ ID NO: 43 - protein, 34 aa, Artificial Sequence
  Description of Artificial Sequence Synthetic polypeptide
YGRKKRRQRRRATGVLYLLVVLVWVAAQGVLTVL